SEQUENCE LISTING (11 sequences)

SEQ ID NO: 1 - protein, 9 aa, Bothrops jararaca
  AMIDATION (9)..(9): The C-terminal is amidated
EWPRPQIPP

SEQ ID NO: 2 - DNA, 24 bp, Artificial Sequence
  Synthetic polynucleotide prepared in the lab
gccctgcaggtgtctgcagcatgt

SEQ ID NO: 3 - DNA, 24 bp, Artificial Sequence
  Synthetic polynucleotide prepared in the lab
ggatggctctccccgccttgtctc

SEQ ID NO: 4 - DNA, 24 bp, Artificial Sequence
  Synthetic polynucleotide prepared in the lab
tgggaccacagcgcccgccactac

SEQ ID NO: 5 - DNA, 24 bp, Artificial Sequence
  Synthetic polynucleotide prepared in the lab
tcgccagccctcccatgcccataa

SEQ ID NO: 6 - DNA, 20 bp, Artificial Sequence
  Synthetic polynucleotide prepared in the lab
agagtgatccaggactgctt

SEQ ID NO: 7 - DNA, 20 bp, Artificial Sequence
  Synthetic polynucleotide prepared in the lab
gttcaggcagctgttgacaa

SEQ ID NO: 8 - DNA, 20 bp, Artificial Sequence
  Synthetic polynucleotide prepared in the lab
aagaagtcccgagaggtgta

SEQ ID NO: 9 - DNA, 20 bp, Artificial Sequence
  Synthetic polynucleotide prepared in the lab
acggagatcgaggttctcaa

SEQ ID NO: 10 - DNA, 21 bp, Artificial Sequence
  Synthetic polynucleotide prepared in the lab
aatgtcgcggaggactttgat

SEQ ID NO: 11 - DNA, 21 bp, Artificial Sequence
  Synthetic polynucleotide prepared in the lab
aggatggcaagggacttcctg

(19) United States
(12) Patent Application Publication
(10) Pub. No.: US 2021/0395309 A1
(43) Pub. Date: Dec. 23, 2021

(54) PEPTIDE-DRUG CONJUGATES

(71) Applicant: Hong Kong Baptist University, Hong Kong (CN)

(72) Inventors: Zhaoxiang BIAN, Hong Kong (CN); Tao HUANG, Hong Kong (CN); Xuanming GUO, Hong Kong (CN); Chengyuan LIN, Hong Kong (CN)

(21) Appl. No.: 17/304,472

(22) Filed: Jun. 22, 2021

Related U.S. Application Data
(60) Provisional application No. 63/043,112, filed on Jun. 23, 2020.

(51) Int. Cl. C07K 7/18; A61K 31/337; A61K 47/64

(52) U.S. Cl. CPC C07K 7/18; A61K 38/00; A61K 47/64; A61K 31/337

(57) ABSTRACT

Provided herein are Bradykinin-potentiating peptide chemotherapeutic drug conjugates useful as cancer therapeutics, pharmaceutical compositions comprising the same, and methods of preparation and use thereof.

### CROSS-REFERENCE TO RELATED APPLICATIONS

This application claims priority from U.S. Provisional Patent Application No. 63/043,112 filed on Jun. 23, 2020, the content of which is hereby incorporated by reference in its entirety.

### TECHNICAL FIELD

The present disclosure generally relates to peptide-drug conjugates (PDC) useful in the treatment of cancer. More particularly, the present disclosure relates to peptide-chemotherapeutic agent conjugates that can be effective and low-toxicity therapies for cancer, such as angiotensin-converting enzyme positive triple-negative breast cancer.

### BACKGROUND

Triple-negative breast cancer (TNBC), representing approximately 15-20% of all invasive breast cancers, is a heterogeneous subtype immunohistochemically characterized to be negative in 3 receptors: estrogen receptor (ER), progesterone receptor (PR), and human epidermal growth factor receptor 2 (HER2). The following features characterize TNBC: onset at a younger age, unique molecular portrait, greater mean tumor size, aggressive nature, a higher tumor grade, distinct metastatic patterns, and a lack of targeted therapies. TNBC is the most difficult breast cancer subtype to treat due to inconsistencies in response to current therapeutic drugs and poor prognosis, high rates of relapse, and frequent metastasis to the visceral and central nervous systems. Current means of treatment encompass surgical resection, chemotherapy, and radiotherapy. In 2018, the PD-L1 inhibitor atezolizumab, combined with nab-paclitaxel, demonstrated improved survival benefits compared with nab-paclitaxel alone in advanced TNBC. Based on these results, atezolizumab plus nab-paclitaxel was approved as a first-line treatment for PD-L1-positive TNBC. However, only 40% of PD-L1-positive TNBC patients can benefit from this therapy, and those who cannot benefit from immunotherapy require novel targeted therapies.

Because of molecular complexity and heterogeneity, TNBC lacks the targeted treatments available for other subtypes of breast cancer. Angiotensin-converting enzyme (ACE) belongs to the type-I membrane-anchored dipeptidylcarboxypeptidase family and controls blood pressure via the renin-angiotensin system (RAS). ACE converts angiotensin I to the active vasoconstrictor angiotensin II, indirectly causing blood vessel contraction and increasing blood pressure. The degradation of bradykinin and other peptides is also carried out by ACE. ACE is distributed in both lung and kidney epithelial cells. ACE was discovered in 1956 and the first crystal structure was published in 2002. There are two functional domains (N- and C-domains) in human ACE, each with a zinc-binding active site. Interestingly, although the N- and C-domains catalyze substrate hydrolysis with similar efficiencies, the inhibition of the N-domain of ACE does not regulate blood pressure. In contrast, inhibiting the C-domain was found to be sufficient for controlling blood pressure. ACE inhibitors are now widely used treatments for cardiovascular diseases. There is some evidence indicating that ACE and other RAS components are mainly expressed in tumor blood vessels of some cancers, including human glioblastoma. It was also observed by Lever et al. that patients treated with ACE inhibitors exhibited reduced incidence of breast and lung cancers. However, these findings were not confirmed by follow-up studies. As opposed to the conflicting patient data, ACE inhibitors do inhibit the growth of many types of tumor cells, including breast cancer cells. These studies thus suggested that ACE is involved in cancer development, but the link between ACE and TNBC is still not clear.

Bradykinin-potentiating peptides (BPPs) are a group of peptides found in Bothrops jararaca venom, which exhibit bradykinin-enhancement effects by inhibiting bradykinin degradation. It has been reported that these bradykinin-potentiating peptides belong to a group of proline-rich oligopeptides whose biological functions are related to inhibition of angiotensin II generation and bradykinin degradation through ACE-inhibition. In hypertensive patients who were administered BPP9a (EWPRPQIPP-NH₂, SEQ ID NO:1) parenterally, the arterial blood pressure dropped significantly. BPPs are potent ACE inhibitors, and structural and functional studies have revealed that BPPs may selectively inhibit the C-domain of ACE.

Paclitaxel (PTX) is a common and potent anti-proliferative chemotherapeutic used against a wide range of malignancies including those of the lung, head and neck, breast and ovary. Various studies have investigated the antitumor effects of paclitaxel in combination with other drugs, showing positive results in TNBC. Results of phase II/III studies indicate that nab-paclitaxel may be effective as a neoadjuvant treatment of TNBC. However, paclitaxel exhibits limitations that include the following: (a) poor aqueous solubility, as low as 0.07 μg/mL, which requires it to be only administered intravenously; (b) poor bioavailability; (c) association with several clinical toxicities, and; (d) can induce drug resistance. The benefits of paclitaxel cytotoxic chemotherapy for TNBC are clear; nevertheless, response rates are low, and over 50% of TNBC patients typically become resistant to paclitaxel chemotherapy within 6-10 months. Therefore, efficient and novel delivery systems for paclitaxel administration are of tremendous interest to the scientific community. These include prodrug strategies, nanoparticle-based delivery systems such as nanoparticle albumin-bound (nab)-paclitaxel that are in various stages of clinical testing, paclitaxel-eluting membranes (PEM), and peptide-carrier methods which include PDCs.

There is thus a need for improved cancer therapeutics that address at least some of the disadvantages described above.

### SUMMARY

Described herein is the design, synthesis and evaluation—in vitro and in vivo—of a novel class of PDC, exemplified by BPP-PTX (PDC of Formula V), in which paclitaxel is conjugated to one member of bradykinin potentiating peptides (BPP), Bj-BPP-9a (teprotide), via a succinyl linker. It is demonstrated that BPP-PTX functions through cognate receptor ACE. ACE was overexpressed in TNBC cell lines, but not in receptor-positive cell line. BPP as part of BPP-PTX bound ACE and mediated its selective cytotoxic action through this receptor in ACE-positive TNBC cells. Furthermore, BPP-PTX demonstrated improved therapeutic activity and a better safety profile in vivo.

In a first aspect, provided herein is A peptide-drug conjugate (PDC) of Formula I:

R¹—X—R² I

or a pharmaceutically acceptable salt or zwitterion thereof, wherein

X is a linker;

R¹ is a chemotherapeutic agent;

R² is a Bradykinin-potentiating polypeptide, wherein the linker is covalently bonded to the N-terminal nitrogen of the Bradykinin-potentiating polypeptide.

In a first embodiment of the first aspect, provided herein is the PDC of the first aspect, wherein the chemotherapeutic agent is a taxane.

In a second embodiment of the first aspect, provided herein is the PDC of the first aspect, wherein the chemotherapeutic agent is paclitaxel, docetaxel, or cabazitaxel.

In a third embodiment of the first aspect, provided herein is the PDC of the first aspect, wherein the Bradykinin-potentiating polypeptide comprises SEQ ID NO:1.

In a fourth embodiment of the first aspect, provided herein is the PDC of the first aspect, wherein the linker is selected from the group consisting of: *—C(═O)—**, *—(CR₂)ₙ—**, *—(CR₂)ₙCHOHCH₂—**, *—(CR₂)ₙC(═O)—**, *—C(═O)(CR₂)ₙ—**, *—C(═O)(CR₂)ₙC(═O)—**, *—C(═O)O(CR₂)ₙ—**, *—(CR₂)ₙOC(═O)—**, *—C(═O)O(CR₂)ₙOC(═O)—**, *—C(═O)N(R)(CR₂)ₙ—**, *—(CR₂)ₙ(R)NC(═O)—**, *—C(═O)(CR₂)ₙ(R)NC(═O)—**, *—C(═O)N(R)(CR₂)ₙC(═O)—**, *—C(═O)N(R)(CR₂)ₙ(R)NC(═O)—**, *—C(═O)O(CR₂)ₙ(R)NC(═O)—**, C(═O)N(R)(CR₂)ₙOC(═O)—**, *—(CR₂)ₙSO₂—**, *—SO₂N(R)(CR₂)ₙ—**, and *—(CR₂)ₙN(R) SO₂—**, wherein * indicates the position of a covalent bond with the chemotherapeutic agent and ** indicates the position of a covalent bond with R²; each instance of n is independently a whole number selected from 1-10; and R for each instance is independently selected from hydrogen, alkyl, cycloalkyl, and aryl; or two instances of R taken together with the carbons to which they are attached form a 3-6 membered carbocylic ring; or two instances of R taken together with the atoms to which they are attached form a 5-6 membered heterocyclic ring.

In a fifth embodiment of the first aspect, provided herein is the PDC of the first aspect, wherein the linker is *—C(═O)—**, *—C(═O)(CR₂)ₙC(═O)—**, *—C(═O)N(R)(CR₂)ₙ(R)NC(═O)—**, or *—C(═O)N(R)(CR₂)ₙC(═O)—**, wherein n is a whole number selected from 2-4.

In a sixth embodiment of the first aspect, provided herein is the PDC of the first aspect, wherein the chemotherapeutic agent has the Formula II:

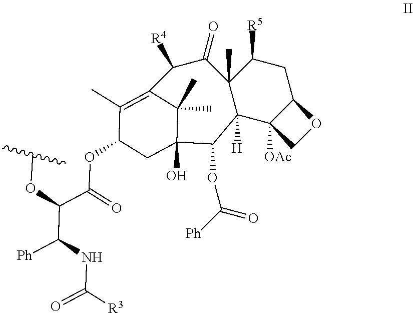

wherein R³ is phenyl, R⁴ is OAc, and R⁵ is OH; R³ is OtBu, R⁴ is OH, and R⁵ is OH; or R³ is OtBu, R⁴ is OMe, and R⁵ is OMe, wherein [symbol] indicates the position of a covalent bond with X.

In a seventh embodiment of the first aspect, provided herein is the PDC of the first aspect, wherein the compound has Formula III:

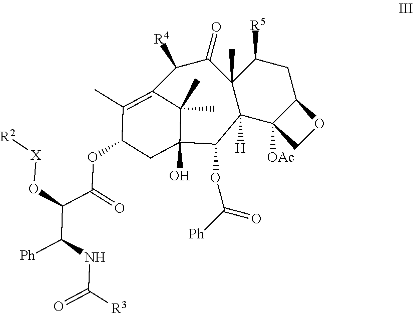

or a pharmaceutically acceptable salt or zwitterion thereof, wherein

X is a linker; and

R² is a Bradykinin-potentiating polypeptide comprising SEQ ID NO:1; and

R³ is phenyl, R⁴ is OAc, and R⁵ is OH; R³ is OtBu, R⁴ is OH, and R⁵ is OH; or R³ is OtBu, R⁴ is OMe, and R⁵ is OMe, wherein the linker is covalently bonded to the N-terminal nitrogen of the Bradykinin-potentiating polypeptide.

In an eighth embodiment of the first aspect, provided herein is the PDC of the seventh embodiment of the first aspect, wherein the linker is selected from the group consisting of: *—(CH₂)ₙ—**, *—(CH₂)ₙCHOHCH₂—**, *—(CH₂)ₙC(═O)—**, *—C(═O)(CH₂)ₙ—**, *—C(═O)(CH₂)ₙC(═O)—**, *—C(═O)O(CH₂)ₙ—**, *—(CH₂)ₙOC(═O)—**, *—C(═O)O(CH₂)ₙOC(═O)—**, *—C(═O)N(H)(CH₂)—**, *—(CH₂)ₙ(H)NC(═O)—**, *—C(═O)N(H)(CH₂)ₙ(H)NC(═O)—**, *—C(═O)O(CH₂)(H)NC(═O)—**, *—C(═O)N(H)(CH₂)ₙOC(═O)—**, *—(CH₂)ₙSO₂—**, *—SO₂N(H)(CH₂)ₙ—**, and *—(CH₂)ₙN(H)SO₂—**, wherein * indicates the position of a covalent bond to a moiety of Formula IV:

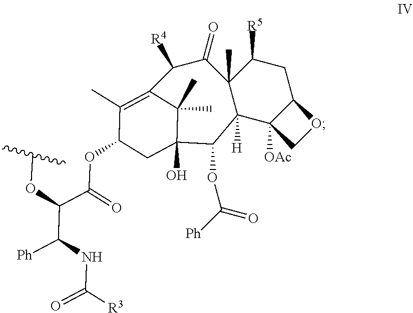

and ** indicates the position of a covalent bond with R²; and each instance of n is independently a whole number selected from 2-6.

In a ninth embodiment of the first aspect, provided herein is the PDC of the eighth embodiment of the first aspect, wherein the linker is *—C(═O)(CR₂)ₙC(═O)—**, wherein n is 2-4.

In a tenth embodiment of the first aspect, provided herein is the PDC of the first aspect, wherein the compound is selected from the group consisting of:

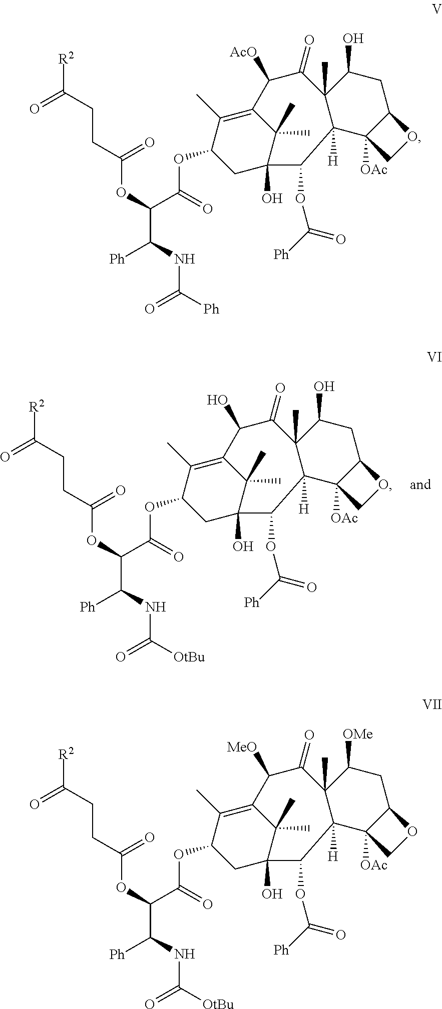

or a pharmaceutically acceptable salt or zwitterion thereof, wherein R² is SEQ ID NO:1.

In an eleventh embodiment of the first aspect, provided herein is the PDC of the first aspect, wherein the compound is:

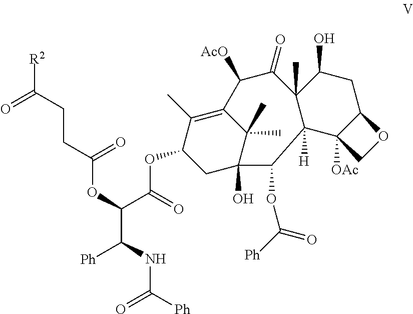

or a pharmaceutically acceptable salt or zwitterion thereof, wherein R² is SEQ ID NO:1.

In a second aspect, provided herein is a pharmaceutical composition comprising a PDC of the first aspect and at least one pharmaceutically acceptable excipient.

In a third aspect, provided herein is a method of preparing the PDC of the eleventh embodiment of the first aspect, the method comprising: contacting a compound of Formula VIII:

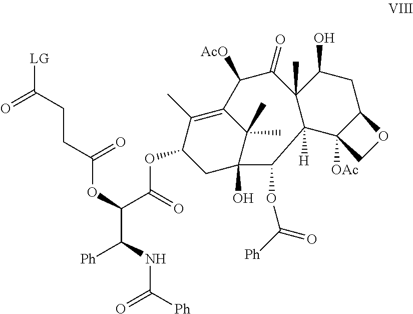

wherein LG is a leaving group; with a polypeptide comprising SEQ ID NO: 1 thereby forming the PDC of the eleventh embodiment of the first aspect.

In a fourth aspect, provided herein is a method of treating cancer in a subject in need thereof, the method comprising: administering a therapeutically effective amount of the PDC of the first aspect to the subject.

In a first embodiment of the fourth aspect, provided herein is the method of the fourth aspect, wherein the cancer is selected from the group consisting of breast cancer, gastric cancer, lung cancer, head cancer, neck cancer, colon cancer, pancreatic cancer, melanoma, brain cancer, human glioblastoma, renal cancer, prostate cancer, and ovarian cancer.

In a second embodiment of the fourth aspect, provided herein is the method of the fourth aspect, wherein the cancer is angiotensin-converting enzyme (ACE) positive.

In a third embodiment of the fourth aspect, provided herein is the method of the fourth aspect, wherein the cancer is breast cancer.

In a fourth embodiment of the fourth aspect, provided herein is the method of the fourth aspect, wherein the cancer is ACE positive triple-negative breast cancer (TNBC).

In a fifth embodiment of the fourth aspect, provided herein is the method of the fourth aspect, wherein the PDC is:

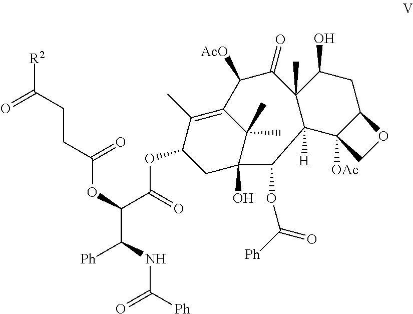

or a pharmaceutically acceptable salt or zwitterion thereof, wherein R² is SEQ ID NO:1.

Other aspects and advantages of the invention will be apparent to those skilled in the art from a review of the ensuing description.

### BRIEF DESCRIPTION OF DRAWINGS

The above and other objects and features of the present invention will become apparent from the following description of the invention, when taken in conjunction with the accompanying drawings, in which:

FIG. 1A shows the expression of ACE in human healthy and breast cancer cell lines. The relative mRNA expression of ACE, ACE2, BR1, and BR2 in different healthy and breast cancer cell lines. For each gene, the level of mRNA expression of the breast cancer cell line was compared with that of HEK293T. *, p<0.05; **, p<0.01; ***, p<0.001; ****, p<0.0001; ns, not significant.

FIG. 1B shows the expression of ACE in human healthy and breast cancer cell lines. The protein expression levels of ACE, ACE2, BR1, and BR2 in different healthy and breast cancer cell lines. For each protein, the normalized expression level (normalized by β-actin) of the breast cancer cell line was compared with that of HEK293T. *, p<0.05; **, p<0.01; ***, p<0.001; ****, p<0.0001; ns, not significant. HEK293T, human embryonic kidney 293 cells along with T antigen of SV40; MCF-7, receptor positive breast cancer cell line; MDA-MB-231, receptor negative breast cancer cell line; MDA-MB-468, receptor negative breast cancer cell line.

FIG. 2 shows the design of BPP-PTX conjugates. The BPP-PTX conjugate is comprised of three parts: the warhead, paclitaxel; the linker, succinyl; and the carrier, BPP9a peptide.

FIG. 3A shows cytotoxicity of BPP-PTX in ACE-negative and ACE-positive cell lines. The cytotoxicity of BPP, PTX, and BPP-PTX in ACE-negative cell line (HEK-293T).

FIG. 3B shows cytotoxicity of BPP-PTX in ACE-negative and ACE-positive cell lines. The cytotoxicity of BPP, PTX, and BPP-PTX in ACE-positive TNBC cell lines (MDA-MB-231).

FIG. 3C shows cytotoxicity of BPP-PTX in ACE-negative and ACE-positive cell lines. The cytotoxicity of BPP, PTX, and BPP-PTX in ACE-positive TNBC cell lines (MDA-MB-468).

FIG. 3D shows cytotoxicity of BPP-PTX in ACE-negative and ACE-positive cell lines. The cytotoxicity of PTX in ACE-negative cell (HEK293T) and ACE-overexpressed counterpart (HEK293T-ACE). *, p<0.05; **, p<0.01; ***, p<0.001; ****, p<0.0001; ns, not significant.

FIG. 3E shows cytotoxicity of BPP-PTX in ACE-negative and ACE-positive cell lines. The cytotoxicity of BPP-PTX in ACE-negative cell (HEK293T) and ACE-overexpressed counterpart (HEK293T-ACE). *, p<0.05; **, p<0.01; ***, p<0.001; ****, p<0.0001; ns, not significant.

FIG. 3F shows cytotoxicity of BPP in ACE-negative and ACE-positive cell lines. The cytotoxicity of PTX in ACE-positive cell (MDA-MB-231-siCtr and ACE-knockdown counterparts (MDA-MB-231-siACE). *, p<0.05; **, p<0.01; ***, p<0.001; ****, p<0.0001; ns, not significant.

FIG. 3G shows cytotoxicity of BPP-PTX in ACE-negative and ACE-positive cell lines. The cytotoxicity of BPP-PTX in ACE-positive cell (MDA-MB-231-siCtr) and ACE-knockdown counterparts (MDA-MB-231-siACE). *, p<0.05; **, p<0.01; ***, p<0.001; ****, p<0.0001; ns, not significant.

FIG. 3H shows cytotoxicity of PTX in ACE-negative and ACE-positive cell lines. The cytotoxicity of PTX in ACE-positive cell (MDA-MB-468-siCtr) and ACE-knockdown counterparts (MDA-MB-468-siACE). *, p<0.05; **, p<0.01; ***, p<0.001; ****, p<0.0001; ns, not significant.

FIG. 3I shows cytotoxicity of BPP-PTX in ACE-negative and ACE-positive cell lines. The cytotoxicity of BPP-PTX in ACE-positive cell (MDA-MB-468-siCtr) and ACE-knockdown counterparts (MDA-MB-468-siACE). *, p<0.05; **, p<0.01; ***, p<0.001; ****, p<0.0001; ns, not significant.

FIG. 4A shows the induced apoptosis in ACE-positive cells incubated with 10 nM BPP, PTX or BPP-PTX for 48 h. The DAPI staining results of apoptosis in PTX and BPP-PTX treated MDA-MB-231 and MDA-MB-468 cells. Cells were fixed and stained with DAPI. Cell images were captured using fluorescence microscopy. Nuclei of apoptotic cells were fragmented and condensed.

FIG. 4B shows the induced apoptosis in ACE-positive cells incubated with 10 nM BPP, PTX or BPP-PTX for 48 h. Apoptotic analysis of ACE-positive cells (MDA-MB-231 and MDA-MB-468) by flow cytometry. The two cell lines were treated with 10 nM BPP, PTX or BPP-PTX and incubated with AV-FITC and PI. Stained cells were analyzed by flow cytometry. Percentage of intact cells (AV−/PI−) and different stages of apoptotic cells (AV+/PI−, AV+/PI+, and AV−/PI+) are presented.

FIG. 4C shows the induced apoptosis in ACE-positive cells incubated with 10 nM BPP, PTX or BPP-PTX for 48 h (MDA-MB-231, receptor negative breast cancer cell line). Quantification of apoptosis data. *, p<0.05; **, p<0.01; ***, p<0.001; ****, p<0.0001; ns, not significant.

FIG. 4D shows the induced apoptosis in ACE-positive cells incubated with 10 nM BPP, PTX or BPP-PTX for 48 h (MDA-MB-468, receptor negative breast cancer cell line). Quantification of apoptosis data. *, p<0.05; **, p<0.01; ***, p<0.001; ****, p<0.0001; ns, not significant.

FIG. 5A show in vivo anticancer efficacy of PTX and BPP-PTX in MDA-MB-468 cell xenografted female nude mice. All drugs were administered by intraperitoneal injection. Photos of tumors separated from mice. Scale bar, 1 cm.

FIG. 5B show in vivo anticancer efficacy of PTX and BPP-PTX in MDA-MB-468 cell xenografted female nude mice. All drugs were administered by intraperitoneal injection. Tumor volume profiles during the 28-day treatment.

FIG. 5C show in vivo anticancer efficacy of PTX and BPP-PTX in MDA-MB-468 cell xenografted female nude mice. Weights of separated tumors from mice.

FIG. 5D show in vivo anticancer efficacy of PTX and BPP-PTX in MDA-MB-468 cell xenografted female nude mice. Mice body weight profiles during the 28-day treatment.

FIG. 5E show in vivo anticancer efficacy of PTX and BPP-PTX in MDA-MB-468 cell xenografted female nude mice. The white blood cell (WBC) count before and after drug treatment in xenografted mice. *, p<0.05; **, p<0.01; ***, p<0.001; ****, p<0.0001; ns, not significant.

FIG. 6A shows an exemplary synthetic route to BPP-PTX. Reagent and condition-a) Succinic anhydride, 4-Dimethylaminopyridine (DMAP), Pyridine, Dichloromethane (DCM), room temperature (r.t), 4 h; b) N-Hydroxysuccinimide, N,N′ Dicyclohexylcarbodiimide (DCC), chloroform (CHCl₃), room temperature (r.t), 6 h; c) R-Peptide, Na₂CO₃ (PH-8), Water (H₂O), Acetone, room temperature 25 (r.t), 10 min to 1 h.

FIG. 6B shows the final product of BPP-PTX synthesis.

FIG. 7 shows BBP (EWPRPQIPP-NH₂, SEQ ID NO:1). A diagram of the peptide synthesis (BPP). Fmoc-Pro-OH (0.135 g), Fmoc-Ile-OH (0.141 g), Fmoc-Gln (Trt)-OH (0.244 g), Fmoc-Arg (pdf)-OH (0.260 g), Fmoc-Trp (Boc)-OH (0.211 g) and Fmoc-Glu (OtBu)-OH (0.170 g), 2-(1H-Benzotriazole-1-yl)-1,1,3,3-tetramethyluronium hexafluorophospha (HBTU) and Rink amide resin (AM), purchased from Top-peptide Co., Reagents including diethyl ether, N,N-diisopropylcarbodiimide, N,N-dimethylformamide (DMF), dichloromethane (DCM), anhydrous ethanol, trifluoroacetic acid (TFA), triethylamine, triisopropylsilane (TIS), piperidine, 1-hydroxybenzotriazole and acetonitrile were purchased from commercial suppliers. The Crude peptide was dried with a Shanghai Tianfeng TF-FD-1 freeze drier, purified by Agilent ProStar HPLC with an SB-C18 column, and analyzed by Agilent Technologies 1260 Infinity HPLC with a kromasil C18-5 column. The LC-MS spectra were determined on an Agilent Technologies 6420 Triple Quad LC/MS.

FIG. 8 shows the high performance liquid chromatography (HPLC) chromatogram of BPP.

### DETAILED DESCRIPTION

### Definitions

The definitions of terms used herein are meant to incorporate the present state-of-the-art definitions recognized for each term in the field of biotechnology. Where appropriate, exemplification is provided. The definitions apply to the terms as they are used throughout this specification, unless otherwise limited in specific instances, either individually or as part of a larger group.

Throughout this specification, unless the context requires otherwise, the word “comprise” or variations such as “comprises” or “comprising”, will be understood to imply the inclusion of a stated integer or group of integers but not the exclusion of any other integer or group of integers. It is also noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

Furthermore, throughout the specification and claims, unless the context requires otherwise, the word “include” or variations such as “includes” or “including”, will be understood to imply the inclusion of a stated integer or group of integers but not the exclusion of any other integer or group of integers.

When trade names are used herein, applicants intend to independently include the trade name product formulation, the generic drug, and the active pharmaceutical ingredient(s) of the trade name product.

The term “amino acid” refers to naturally occurring and non-natural amino acids, as well as amino acid analogs and amino acid mimetics that function in a manner similar to the naturally occurring amino acids. Naturally encoded amino acids are the 20 common amino acids (alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine) and pyrrolysine and selenocysteine. Amino acid analogs refers to compounds that have the same basic chemical structure as a naturally occurring amino acid, i.e., an α carbon that is bound to a hydrogen, a carboxyl group, an amino group, and an R group, such as, homoserine, norleucine, methionine sulfoxide, methionine methyl sulfonium. Such analogs have modified R groups (such as, norleucine) or modified peptide backbones, but retain the same basic chemical structure as a naturally occurring amino acid.

A “non-natural amino acid” refers to an amino acid that is not one of the 20 common amino acids or pyrrolysine or selenocysteine; other terms that may be used synonymously with the term “non-natural amino acid” is “non-naturally encoded amino acid,” “unnatural amino acid,” “non-naturally-occurring amino acid,” and the like. The term “non-natural amino acid” includes, but is not limited to, amino acids that have the same basic chemical structure as a naturally occurring amino acid, i.e., an α carbon that is bound to a hydrogen, a carboxyl group, an amino group, and an R group, as well as amino acids that amino acids in which the amino group is attached at the β or γ carbon.

Amino acid substitutions of the described polypeptides can be conservative amino acid substitutions. Conservative amino acid substitutions are known in the art, and include amino acid substitutions in which one amino acid having certain physical and/or chemical properties is exchanged for another amino acid that has the same or similar chemical or physical properties. For instance, the conservative amino acid substitution can be an acidic/negatively charged polar amino acid substituted for another acidic/negatively charged polar amino acid (e.g., Asp or Glu), an amino acid with a nonpolar side chain substituted for another amino acid with a nonpolar side chain (e.g., Ala, Gly, Val, Ile, Leu, Met, Phe, Pro, Trp, Cys, Val, etc.), a basic/positively charged polar amino acid substituted for another basic/positively charged polar amino acid (e.g. Lys, His, Arg, etc.), an uncharged amino acid with a polar side chain substituted for another uncharged amino acid with a polar side chain (e.g., Asn, Gln, Ser, Thr, Tyr, etc.), an amino acid with a beta-branched side-chain substituted for another amino acid with a beta-branched side-chain (e.g., Ile, Thr, and Val), an amino acid with an aromatic side-chain substituted for another amino acid with an aromatic side chain (e.g., His, Phe, Trp, and Tyr), etc.

The terms “percentage homology” and “percentage sequence identity”, when used in reference to a polypeptide or polynucleotide sequence, are used interchangeably herein to refer to comparisons among polynucleotides and polypeptides, and are determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. Homology is evaluated using any of the variety of sequence comparison algorithms and programs known in the art. Such algorithms and programs include, but are by no means limited to, TBLASTN, BLASTP, FASTA, TFASTA, and CLUSTALW [Pearson and Lipman, 1988, Proc. Natl. Acad. Sci. USA 85(8):2444-2448; Altschul et al., 1990, J. Mol. Biol. 215(3):403-410; Thompson et al., 1994, Nucleic Acids Res. 22(2):4673-4680; Higgins et al. 1996, Methods Enzymol. 266:383-402; Altschul et al., 1990, J. Mol. Biol. 215(3):403-410; Altschul et al., 1993, Nature Genetics 3:266-272]. In certain embodiments, protein and nucleic acid sequence homologies are evaluated using the Basic Local Alignment Search Tool (“BLAST”) which is well known in the art (see, e.g., Karlin and Altschul, 1990, Proc. Natl. Acad. Sci. USA 87:2267-2268; Altschul et al., 1990, J. Mol. Biol. 215:403-410; Altschul et al., 1993, Nature Genetics 3:266-272; Altschul et al., 1997, Nuc. Acids Res. 25:3389-3402).

The term “alkyl” is art-recognized, and includes saturated aliphatic groups, including straight-chain alkyl groups, branched-chain alkyl groups, cycloalkyl (alicyclic) groups, alkyl substituted cycloalkyl groups, and cycloalkyl substituted alkyl groups. In certain embodiments, a straight chain or branched chain alkyl has about 30 or fewer carbon atoms in its backbone (e.g., C₁-C₃₀ for straight chain, C₃-C₃₀ for branched chain), and alternatively, about 20 or fewer. Likewise, cycloalkyls have from about 3 to about 10 carbon atoms in their ring structure, and alternatively about 5, 6 or 7 carbons in the ring structure.

The term “aryl” is art-recognized and refers to 5-, 6- and 7-membered single-ring aromatic groups that may include from zero to four heteroatoms, for example, benzene, naphthalene, anthracene, pyrene, pyrrole, furan, thiophene, imidazole, oxazole, thiazole, triazole, pyrazole, pyridine, pyrazine, pyridazine and pyrimidine, and the like. Those aryl groups having heteroatoms in the ring structure may also be referred to as “aryl heterocycles” or “heteroaromatics.” The aromatic ring may be substituted at one or more ring positions with such substituents as described above, for example, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, —CN, or the like. The term “aryl” also includes polycyclic ring systems having two or more cyclic rings in which two or more carbons are common to two adjoining rings (the rings are “fused rings”) wherein at least one of the rings is aromatic, e.g., the other cyclic rings may be cycloalkyls, cycloalkenyls, cycloalkynyls, aryls and/or heterocyclyls.

The representation “ [symbol] ” as used herein in connection to chemical a group or moiety is intended to represent the covalent bond that the aforementioned chemical group or moiety is covalently bonded to another chemical group, moiety, or compound.

As used herein, the term “pharmaceutically acceptable salt” refers to those salts which are, within the scope of sound medical judgment, suitable for use in contact with the tissues of subjects without undue toxicity, irritation, allergic response and the like, and are commensurate with a reasonable benefit/risk ratio. Pharmaceutically acceptable salts are well known in the art. For example, Berge et al. describes pharmaceutically acceptable salts in detail in J. Pharmaceutical Sciences (1977) 66:1-19. Pharmaceutically acceptable salts of the compounds provided herein include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, besylate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemi sulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. In certain embodiments, organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like.

Pharmaceutically acceptable salts derived from appropriate bases include alkali metal, alkaline earth metal, ammonium and N⁺(C₁₋₄ alkyl)₄ salts. Representative alkali or alkaline earth metal salts include sodium, lithium, potassium, calcium, magnesium, iron, zinc, copper, manganese, aluminum, and the like. Further pharmaceutically acceptable salts include, when appropriate, non-toxic ammonium, quaternary ammonium, and amine cations formed using counter ions, such as halide, hydroxide, carboxylate, sulfate, phosphate, nitrate, lower alkyl sulfonate, and aryl sulfonate. Organic bases from which salts can be derived include, for example, primary, secondary, and tertiary amines, substituted amines including naturally occurring substituted amines, cyclic amines, basic ion exchange resins, and the like, such as isopropylamine, trimethylamine, diethylamine, triethylamine, tripropylamine, and ethanolamine. In certain embodiments, the pharmaceutically acceptable base addition salt is chosen from ammonium, potassium, sodium, calcium, and magnesium salts.

A “chemotherapeutic agent” is a chemical compound useful in the treatment of cancer, regardless of mechanism of action. Classes of chemotherapeutic agents include, but are not limited to: alkyating agents, antimetabolites, spindle poison plant alkaloids, cytotoxic/antitumor antibiotics, topoisomerase inhibitors, antibodies, photosensitizers, and kinase inhibitors. Chemotherapeutic agents include compounds used in “targeted therapy” and conventional chemotherapy.

As used herein, the terms “treat”, “treating”, “treatment”, and the like refer to reducing or ameliorating a disorder/disease and/or symptoms associated therewith. It will be appreciated, although not precluded, treating a disorder or condition does not require that the disorder, condition, or symptoms associated therewith be completely eliminated. In certain embodiments, treatment includes prevention of a disorder or condition, and/or symptoms associated therewith. The term “prevention” or “prevent” as used herein refers to any action that inhibits or at least delays the development of a disorder, condition, or symptoms associated therewith. Prevention can include primary, secondary and tertiary prevention levels, wherein: a) primary prevention avoids the development of a disease; b) secondary prevention activities are aimed at early disease treatment, thereby increasing opportunities for interventions to prevent progression of the disease and emergence of symptoms; and c) tertiary prevention reduces the negative impact of an already established disease by restoring function and reducing disease-related complications.

As used herein, the term “subject” refers to any animal (e.g., a mammal), including, but not limited to, humans, non-human primates, canines, felines, and rodents.

Other definitions for selected terms used herein may be found within the detailed description of the invention and apply throughout. Unless otherwise defined, all other technical terms used herein have the same meaning as commonly understood to one of ordinary skill in the art to which the invention pertains.

PDC are a novel class of therapeutics gaining increasing attention due to easier manufacturing and improved tumor tissue penetration. The architectural elements comprising a PDC include a chemotherapeutic agent, a connecting linker, and a tumor-targeting peptide. The tumor-targeting peptide maneuvers the PDC within the body and zeroes in on its ectopically expressed cognate receptor on tumor cell surfaces, enabling the PDC to deliver its cytotoxic payload to the targeted tumor cell. This efficient delivery strategy enhances the therapeutic effect and drastically reduces damage to healthy cells. PDCs have shown promising results in pre-clinical and clinical trials for cancer therapy.

The PDC described herein can be represented by the Formula I:

R¹—X—R² I

or a pharmaceutically acceptable salt or zwitterion thereof, wherein

X is a linker;

R¹ is a chemotherapeutic agent;

R² is a Bradykinin-potentiating polypeptide, wherein the linker is covalently bonded to the N-terminal nitrogen of the Bradykinin-potentiating polypeptide.

R¹ can be any chemotherapeutic agent known to those skilled in the art including, but not limited to erlotinib (TARCEVAR®, Genentech/OSI Pharm.), docetaxel (TAXOTERE®, Sanofi-Aventis), 5-FU (fluorouracil, 5-fluorouracil, CAS No. 51-21-8), gemcitabine (GEMZAR®), Lilly), PD-0325901 (CAS No. 391210-10-9, Pfizer), cisplatin (cis-diamine, dichloroplatinum(II), CAS No. 15663-27-1), carboplatin (CAS No. 41575-94-4), paclitaxel (TAXOL®, Bristol-Myers Squibb Oncology, Princeton, N.J.), trastuzumab (HERCEPTIN®, Genentech), temozolomide (4-methyl-5-oxo-2,3,4,6,8-pentazabicyclo[4.3.0]nona-2,7,9-triene-9-carboxamide, CAS No. 85622-93-1, TEMODAR®, TEMODAL®, Schering Plough), tamoxifen ((Z)-2-[4-(1,2-diphenylbut-1-enyl) phenoxy]-N,N-dimethyl-ethanamine, NOLVADEX®, ISTUBAL®, VALODEX®), and doxorubicin (ADRIAMYCIN®), Akti-1/2, HPPD, and rapamycin.

Additional examples of suitable chemotherapeutic agents include: oxaliplatin (ELOXATIN®, Sanofi), bortezomib (VELCADE®, Millennium Pharm.), sutent (SUNITINIB®, SU1 1248, Pfizer), letrozole (FEMARA®, Novartis), imatinib mesylate (GLEEVEC®, Novartis), XL-518 (Mek inhibitor, Exelixis, WO 2007/044515), ARRY-886 (Mek inhibitor, AZD6244, Array BioPharma, Astra Zeneca), SF-1 126 (PI3K inhibitor, Semafore Pharmaceuticals), BEZ-235 (PI3K inhibitor, Novartis), XL-147 (PI3K inhibitor, Exelixis), PTK787/ZK 222584 (Novartis), fulvestrant (FASLODEX®, AstraZeneca), leucovorin (folinic acid), rapamycin (sirolimus, RAPAMUNE®, Wyeth), lapatinib (TYKERB®, GSK572016, Glaxo Smith Kline), lonafarnib (SARASAR™, SCH 66336, Schering Plough), sorafenib (NEXAVAR®, BAY43-9006, Bayer Labs), gefitinib ORES SA®, AstraZeneca), irinotecan (CAMPTOSAR®, CPT-11, Pfizer), tipifarnib (ZARNESTRA™, Johnson & Johnson), ABRAXANE™ (Cremophor-free), albumin-engineered nanoparticle formulations of paclitaxel (American Pharmaceutical Partners, Schaumberg, Ill.), vandetanib (rINN, ZD6474, ZACTIMA®, AstraZeneca), chloranmbucil, AG1478, AG1571 (SU 5271; Sugen), temsirolimus (TORISEL®, Wyeth), pazopanib (GlaxoSmithKline), canfosfamide (TELCYTA®, Telik), thiotepa and cyclosphosphamide (CYTOXAN®, NEOSAR®); alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); a camptothecin (including the synthetic analog topotecan); bryostatin; callystatin; CC-1065 (including its adozelesin, carzelesin and bizelesin synthetic analogs); cryptophycins (particularly cryptophycin 1 and cryptophycin 8); dolastatin; duocarmycin (including the synthetic analogs, KW-2189 and CB1-TM1); eleutherobin; pancratistatin; a sarcodictyin; spongistatin; nitrogen mustards such as chlorambucil, chlomaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; antibiotics such as the enediyne antibiotics (e.g., calicheamicin, calicheamicin gammalI, calicheamicin omegalI (Angew Chem. Intl. Ed. Engl. (1994) 33:183-186); dynemicin, dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfomithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; 6-thioguanine; mercaptopurine; methotrexate; platinum analogs such as cisplatin and carboplatin; vinblastine; etoposide (VP-16); ifosfamide; mitoxantrone; vincristine; vinorelbine (NAVELBINE®); novantrone; teniposide; edatrexate; daunomycin; aminopterin; capecitabine (XELODA®, Roche); ibandronate; CPT-11; topoisomerase inhibitor RFS 2000; difluoromethylornithine (DMFO); retinoids such as retinoic acid; and pharmaceutically acceptable salts, acids and derivatives of any of the above.

In certain embodiments, the chemotherapeutic agent is a taxane selected from the group consisting of is paclitaxel, docetaxel, and cabazitaxel. In certain embodiments, the taxane is albumin-bound.

The Bradykinin-potentiating polypeptide can be BPP10a, BPP6a, BPP13a, BPP10c, BPP10c-F, BPP11b, BPP11b, BPP5a, BPP12b, BPP-APL, BPP11e, BPP11eAP, BPP-AP, BPP-A-AK, BPP-C-AK, BPP-B-AK, BPP-Ahb1, BPP-Ahb2, BPP-1LM, BPP-2LM, BPP-3LM, BPP-4LM, BPP-5LM, BPP12c, BPP-14a, BPP-Tf1, BPP-Tf2, BPP-Tf3, BPP-2-Sisca, BPP-Cdt1a, BPP-Cdt1b, BPP-Cdt2, BPP-Cdt3, BPP-1CRO, BPP-2CRO, BPP-10b, BPP-Tg1, BPP12a, BPP7a, BPP-S, BPP-1-Glo, BPP-11b-CROA, BPP-11c-CROV, BPP-11-BOTAL, BPP11, BPP13a, BPP-2-Glo, BPP-POL-236, BPP7b, BPP7c, BPP8a, BPP9a, BPP10d, BPP10e, BPP10f, BPP11d, BPP11f, BPP11g, BPP11h, BPP11i, BPP11j, BPP-Pb, BPP12a, BPP12b, BPP12d, BPP12e, BPP13a, BPP13b, BPP13c, BPP13d, BPP-TmF, BPP-Phypo-Xa, BPP-VIPAS, BPP-5b-9a-F, BPP-10b-F, BPP-11a-F, BPP-F-AK, BPP-S412, BPP-S51, BPP-11a, or BPP-11b-CRO.

In certain embodiments, the Bradykinin-potentiating polypeptide is BPP5a or BPP9a (SEQ ID NO:1). In certain embodiments, R² is a Bradykinin-potentiating polypeptide is SEQ ID NO:1.

The C-terminal of SEQ ID NO:1 can be —C(═O)NH₂ (amidated) or —CO₂H.

The linker can be *—C(═O)—**, *—(CR₂)ₙ—**, *—(CR₂)ₙCHOHCH₂—**, *—(CR₂)ₙC(═O)—**, *—C(═O)(CR₂)ₙ—**, *—C(═O)(CR₂)ₙC(═O)—**, *—C(═O)O(CR₂)ₙ—**, *—(CR₂)ₙOC(═O)—**, *—C(═O)O(CR₂)ₙOC(═O)—**, *—C(═O)N(R)(CR₂)ₙ—**, *—(CR₂)ₙ(R)NC(═O)—**, *—C(═O)(CR₂)ₙ(R)NC(═O)—**, *—C(═O)N(R)(CR₂)ₙC(═O)—**, *—C(═O)N(R)(CR₂)ₙ(R)NC(═O)—**, *—C(═O)O(CR₂)ₙ(R)NC(═O)—**, *—C(═O)N(R)(CR₂)ₙOC(═O)—**, *—(CR₂)ₙSO₂—**, *—SO₂N(R)(CR₂)ₙ—**, or *—(CR₂)ₙN(R)SO₂—**, wherein * indicates the position of a covalent bond with the chemotherapeutic agent and ** indicates the position of a covalent bond with R²; each instance of n is independently a whole number selected from 1-10; and R for each instance is independently selected from hydrogen, alkyl, cycloalkyl, and aryl; or two instances of R taken together with the carbons to which they are attached form a 3-6 membered carbocylic ring; or two instances of R taken together with the atoms to which they are attached form a 5-6 membered heterocyclic ring.

In certain embodiments, n is a whole number selected from 1-10, 1-8, 1-6, 1-4, 2-4, or 1-3. In certain embodiments, each instance of R is hydrogen.

In certain embodiments, the link is selected from the group consisting of: *—C(═O)—**, *—C(═O)(CR₂)ₙC(═O)—**, *—C(═O)N(R)(CR₂)ₙ(R)NC(═O)—**, *—C(═O)(CR₂)ₙ(R)NC(═O)—**, and *—C(═O)N(R)ₙ(CR₂)C(═O)—**. In certain embodiments, the link is selected from the group consisting of: *—C(═O)—**, *—C(═O)(CH₂)ₙC(═O)—**, *—C(═O)N(R)(CH₂)ₙ(R)NC(═O)—**, *—C(═O)(CH₂)ₙ(R)NC(═O)—**, and *—C(═O)N(R)(CH₂)ₙC(═O)—**, wherein n is 1-6, 1-4, 2-4, or 1-3; and R for each instance is independently hydrogen or alkyl.

The taxane can be conjugated to the linker at any suitably functionalized position of the taxane. For example, the linker can be conjugated via the C-1 hydroxyl, the C-7 hydroxyl, or the side chain beta-hydroxyl. In certain embodiments, the taxane is conjugated to the linker via the side chain beta-hydroxyl as depicted in the moiety of Formula III:

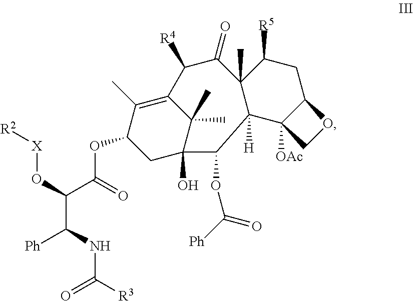

or a pharmaceutically acceptable salt or zwitterion thereof, wherein X is a linker; R² is a polypeptide comprising SEQ ID NO:1; and R³ is phenyl, R⁴ is OAc, and R⁵ is OH; R³ is OtBu, R⁴ is OH, and R⁵ is OH; or R³ is OtBu, R⁴ is OMe, and R⁵ is OMe.

In certain embodiments, the PDC has the Formula V, VI, or VII:

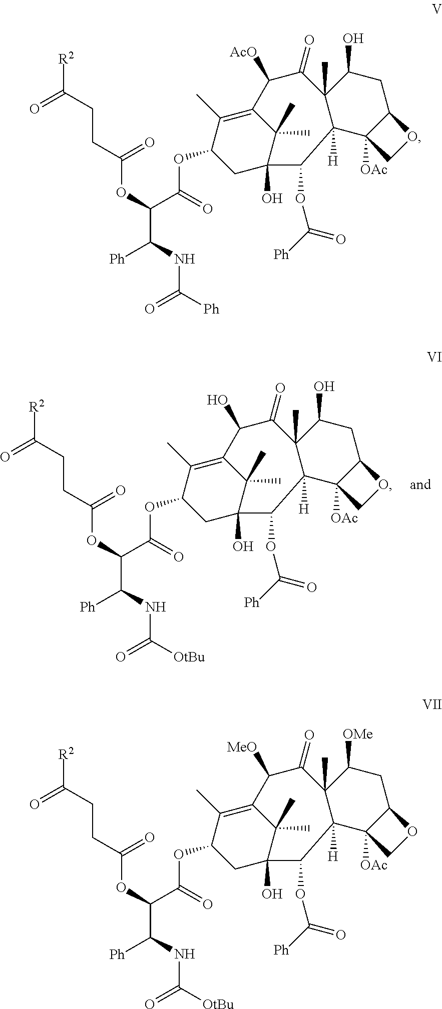

or a pharmaceutically acceptable salt or zwitterion thereof, wherein R² is SEQ ID NO:1.

The present disclosure also provides a pharmaceutical composition comprising a PDC described herein and at least one pharmaceutically acceptable excipient.

The PDC described herein and their pharmaceutically acceptable salts can be administered to a subject either alone or in combination with pharmaceutically acceptable, excipients, carriers, and/or diluents in a pharmaceutical composition according to standard pharmaceutical practice. The PDC can be administered orally or parenterally. Parenteral administration includes intravenous, intramuscular, intraperitoneal, subcutaneous and topical, the preferred method being intravenous and topical administrations.

Accordingly, the present disclosure provides pharmaceutically acceptable compositions, which comprise a therapeutically effective amount of one or more of the PDC described herein, formulated together with one or more pharmaceutically, excipients, acceptable carriers (additives) and/or diluents. The pharmaceutical compositions of the present disclosure may be specially formulated for administration in solid or liquid form, including those adapted for the following: (1) parenteral administration, for example, by subcutaneous, intramuscular, intravenous or epidural injection as, for example, a sterile solution or suspension, or sustained-release formulation; and (2) oral administration, for example, drenches (aqueous or non-aqueous solutions or suspensions), tablets, e.g., those targeted for buccal, sublingual, and systemic absorption, boluses, powders, granules, pastes for application to the tongue.

As set out herein, certain embodiments of the PDC described herein may contain a basic functional group, such as amino, and are, thus, capable of forming pharmaceutically acceptable salts with pharmaceutically acceptable acids. The term “pharmaceutically acceptable salts” in this respect, refers to the relatively non-toxic, inorganic and organic acid addition salts of compounds of the present disclosure. These salts can be prepared in situ in the administration vehicle or the dosage form manufacturing process, or by separately reacting a purified PDC of the invention in its free base form with a suitable organic or inorganic acid, and isolating the salt thus formed during subsequent purification. Representative salts include the hydrobromide, hydrochloride, sulfate, bisulfate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like.

The pharmaceutically acceptable salts of the PDC of the present disclosure include the conventional non-toxic salts or quaternary ammonium salts of the compounds, e.g., from non-toxic organic or inorganic acids. For example, such conventional non-toxic salts include those derived from inorganic acids such as hydrochloride, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane di sulfonic, oxalic, isothionic, and the like.

In other cases, the PDC described herein may contain one or more acidic functional groups and, thus, are capable of forming pharmaceutically acceptable salts with pharmaceutically acceptable bases. The term “pharmaceutically acceptable salts” in these instances refers to the relatively non-toxic, inorganic and organic base addition salts of the PDC of the present disclosure. These salts can likewise be prepared in situ in the administration vehicle or the dosage form manufacturing process, or by separately reacting the purified compound in its free acid form with a suitable base, such as the hydroxide, carbonate or bicarbonate of a pharmaceutically-acceptable metal cation, with ammonia, or with a pharmaceutically-acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like.

Wetting agents, emulsifiers and lubricants, such as sodium lauryl sulfate and magnesium stearate, as well as coloring agents, release agents, coating agents, sweetening, flavoring and perfuming agents, preservatives, solubilizing agents, buffers and antioxidants can also be present in the compositions.

Methods of preparing these formulations include the step of bringing into association a PDC described herein with the carrier or excipient and, optionally, one or more accessory ingredients. In general, the formulations are prepared by uniformly and intimately bringing into association a PDC of the present disclosure with liquid carriers (liquid formulation), liquid carriers followed by lyophilization (powder formulation for reconstitution with sterile water or the like), or finely divided solid carriers, or both, and then, if necessary, shaping or packaging the product.

Pharmaceutical compositions of the present disclosure suitable for parenteral administration comprise one or more PDC described herein in combination with one or more pharmaceutically-acceptable sterile isotonic aqueous or non-aqueous solutions, dispersions, suspensions or emulsions, or sterile powders which may be reconstituted into sterile injectable solutions or dispersions just prior to use, which may contain sugars, alcohols, antioxidants, buffers, bacteriostats, chelating agents, solutes which render the formulation isotonic with the blood of the intended recipient or suspending or thickening agents.

Examples of suitable aqueous and non-aqueous carriers which may be employed in the pharmaceutical compositions of the disclosure include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants, such as preservatives, wetting agents, emulsifying agents and dispersing agents. Prevention of the action of microorganisms upon the PDC of the present disclosure may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption, such as aluminum monostearate and gelatin.

The present disclosure also provides a method of preparing a PDC of Formula V:

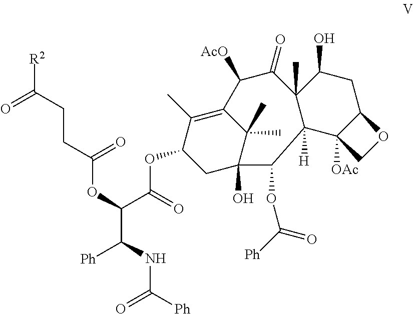

wherein R² is SEQ ID NO:1, the method comprising: contacting a compound of Formula VIII:

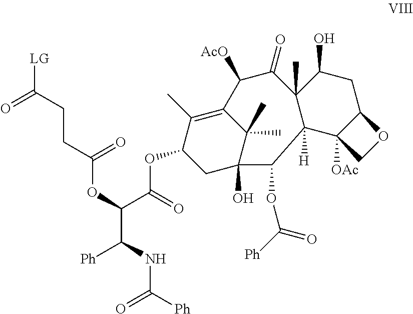

wherein LG is a leaving group; with a polypeptide comprising SEQ ID NO: 1 thereby forming the PDC of Formula V.

The type of leaving group is not particularly limited. Any suitably reactive leaving group can be used in connection with the methods described herein. In certain embodiments, the leaving group is selected from the group consisting of: Cl, Br, I,

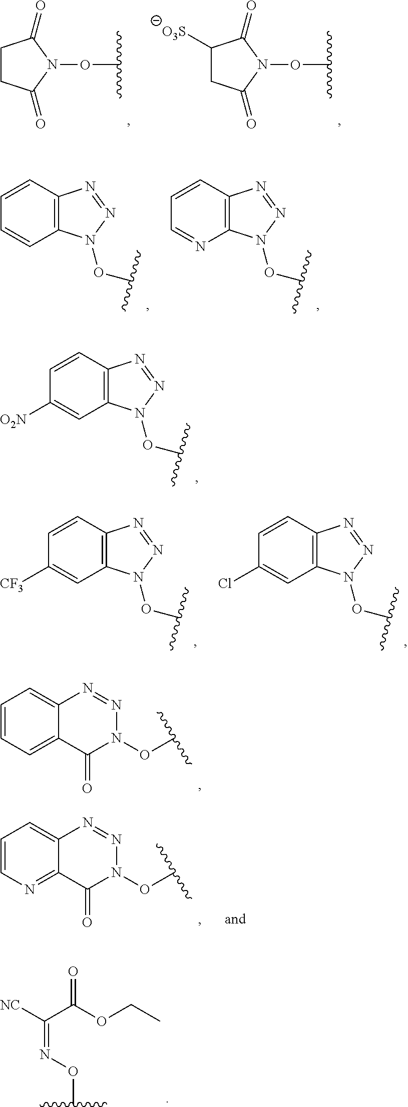

The compound of Formula VIII can be preformed or formed in situ, e.g., by reaction of the corresponding acid with a carbonyl activating agent and optionally a coupling additive.

Exemplary carbonyl activating agents include, but are not limited to, carbodiimide, such as DCC, DIC, EDC, CIC, BMC, CPC, BDDC, PIC, PEC, and BEM, a uronium/aminium salt, such as HATU, HBTU, TATU, TBTU, HAPyU, TAPipU, HAPipU, HBPipU, HAMBU, HBMDU, HAMTU, 5,6-B(HATU), 4,5-B(HATU), HCTU, TCTU, and ACTU, phosphonium salts, such as AOP, BOP, PyAOP, PyBOP, PyOxm, PyNOP, PyFOP, NOP, and PyClock, immonium salts, such as BOMI, BDMP, BMMP, BPMP, and AOMP.

Exemplary coupling additives include, but are not limited to, HOBt. 6-NO₂—HOBt, 6-Cl-HOBt, 6-CF₃—HOBt, HOAt, HODhbt, HODhat, HOSu, and Oxyma.

The preparation of the compound of Formula VIII, including the appropriate selection of reagents and reaction conditions, is well within the skill of a person of ordinary skill in the art.

The step of contacting contacting the compound of Formula VIII SEQ ID NO:1 can take place in any solvent. The selection of the appropriate solvent can depend on the chemical structure of the starting materials and the nature of the chemical reaction required to prepare the compound described herein. The selection of the appropriate solvent is well within the skill of a person of ordinary skill in the art. The solvent can be an aprotic or protic organic solvent. In certain embodiments, the solvent is a polar aprotic solvent. Exemplary solvents include, but are not limited to, water, alcohols, alkyl halides, ethers, esters, ketones, formamides, alkylnitriles, alkylsulfoxides, and aromatic solvents. Exemplary solvents include, but are not limited to, water, tetrahydrofuran, tetrahydropyran, dioxane, dichloromethane, dichloroethane, chloroform, dimethylformamide, dimethylsulfoxide, and mixtures thereof.

The step of contacting contacting the compound of Formula VIII with SEQ ID NO:1 can be conducted in the presence of a base. The base can be an organic or inorganic Brønsted base. In certain embodiments, the base is an organic amine, metal hydroxide, metal carbonate, metal alkoxide, and mixtures thereof. Exemplary bases include, but are not limited to, Hunig's base, pyridine, pyrazine, trimethylamine, morpholine, N-methyl morpholine, piperdine, piperazine, pyrrolidine, DABCO, quinuclidine, TBD, DBU, DBN, DMAP, NaOH, CsOH, KOH, Na₂CO₃, K₂CO₃, Cs₂CO₃, and the like.

The methods described herein can comprise one or more additional synthetic steps before or after the steps described herein. Such synthetic steps may be necessary to prepare more structurally complex PDC of Formula I. The one or more additional synthetic steps can include, but are not limited to, reductions, oxidations, substitution reactions, metal catalysed carbon-carbon forming reactions, alkylations, acylations, electrophilic aromatic substitution reactions, nucleophilic aromatic substitution reactions, hydrolysis reactions, condensation reactions, and the like.

The PDC described herein are useful in the treatment of cancer in a subject in need thereof. The cancer can be a cancer of the head, neck, eye, mouth, throat, esophagus, bronchus, larynx, pharynx, chest, bone, lung, colon, rectum, stomach, prostate, urinary bladder, uterine, cervix, breast, ovaries, testicles or other reproductive organs, skin, thyroid, blood, lymph nodes, kidney, liver, pancreas, and brain or central nervous system. In certain embodiments, the cancer expresses ACE.

In certain embodiments, the cancer is selected from the group consisting of breast cancer, gastric cancer, lung cancer, head cancer, neck cancer, colon cancer, pancreatic cancer, melanoma, brain cancer, renal cancer, prostate cancer, and ovarian cancer.

In certain embodiments, the breast cancer is ACE and TNBC positive.

Reagents

Complete Protease Inhibitor Cocktail Tablets (4693116001) and Phosphatase Inhibitor Cocktail Tablets (4906837001) were obtained from Roche. SuperSignal West Pico Chemiluminescent Substrate (34080) was purchased from Thermo Scientific. The annexin V-FITC Apoptosis Assay Kit (556547) was obtained from BD Pharmingen™. Antibodies against Angiotensin-converting enzyme 1 (ACE) (ab75762), Angiotensin-converting enzyme 2 (ACE2) (ab15348), Bradykinin receptor 1 (BR1) (ab75148), and Bradykinin receptor 2 (BR2) (ab236093) were obtained from Abcam (Cambridge, Mass., USA). β-actin antibody (3700), HRP-goat anti-rabbit secondary antibody (7074), and Goat anti-mouse IgG-HRP secondary antibody (7076) were purchased from Cell Signaling Technology (Danvers, Mass.).

Cell Culture

HEK293T, MCF-7, MDA-MB-231 and MDA-MB-468 cells were purchased from American Type Culture Collection (Manassas, USA). Cells were cultured in DMEM or RPMI supplemented with 10% FBS in a humidified atmosphere containing 5% CO₂ and 95% air at 37° C. The medium was changed every three days, and cells were passaged using 0.05% trypsin/EDTA.

### EXAMPLES

1. Synthesis of 2-Succinyl Paclitaxel

Paclitaxel (200 mg, 0.23 mmol) was added to succinic anhydride (93.78 mg, 0.93 mmol) in the presence of 4-dimethyl amino-pyridine (28.60 mg, 0.23 mmol) which was previously dried under vacuum for 2 h. Then 2 mL of dry pyridine and 2 mL dichloromethane (DCM) was added and the solution was stirred for 3 h at room temperature 25° C. The 2-succinyl paclitaxel was purified by extraction according to the following procedure: After 20 mL of dichloromethane (DCM) was added into the reaction mixture, the organic phase was washed using 1 N HCL solution (20 mL×3). The organic phase was combined and washed with brine (3×10 ml) and dried over Na₂SO₄. The filtrate was concentrated using a rotary evaporator and the crude product was used for the next reaction without purification. white solid, a) yield=87.4%, b) melting point—° C., c) HRMS (ESI): m/z [M+H]+ calcd for 953.34700. Found 976.3369 [Na]+. d) ¹H NMR, CD₃OD, 400 MHz: δ_{H} 8.13 (s, 1H), 8.12 (s, 1H), 7.85 (s, 1H), 7.83 (s, 1H), 7.71 (t, J=7.3 Hz, 1H), 7.66-7.43 (m, 9H), 7.27 (t, J=7.1 Hz, 1H), 6.46 (s, 1H), 6.06 (t, J=8.92 Hz, 1H), 5.81 (d, J=6.7 Hz, 1H), 5.65 (d, J=7.1 Hz, 1H), 5.50 (d, J=5.04 Hz, 1H), 5.02 (d, J=9.0 Hz, 1H), 4.35 (dd, J=6.6 Hz, 10.8 Hz, 1H), 4.20 (s, 2H), 3.82 (d, J=7.2 Hz, 1H), 2.77-2.61 (m, 4H), 2.56-2.48 (m, 1H), 2.40 (s, 3H), 2.19 (s, 3H), 2.16-1.12 (m, 1H), 1.92 (s, 3H), 1.87-1.74 (m, 2H), 1.66 (s, 3H), 1.15 (s, 3H), 1.14 (s, 3H), ¹³C NMR, CD₃OD, 100 MHz: δ_{C} 206.07, 176.63, 174.34, 172.46, 172.17, 171.39, 171.29, 168.49, 143.27, 139.14, 136.28, 135.58, 135.51, 133.76, 132.14, 132.04, 130.93, 130.57, 130.41, 129.48, 129.44, 86.71, 83.02, 79.82, 78.26, 77.62, 77.01, 76.77, 73.72, 73.10, 59.99, 56.12, 48.69, 45.36, 38.30, 37.15, 30.56, 30.41, 27.72, 24.11, 23.20, 21.65, 15.79, and 11.31.

2. Synthesis of 2-NHS-succinyl-paclitaxel

2-succinyl paclitaxel (223.8 mg, 0.23 mmol) was mixed with N-hydroxysucciniimide (32.2 mg, 0.28 mmol) and then 7 mL of chloroform (CHCl3) was added to obtain a well dispersed solution. N, N-dicyclohexylcarbodiimide (57.82 mg, 0.28 mmol) was added into the above mixture and the solution was stirred for 4.30 h at room temperature 25° C. The 2-NHS-succinyl-paclitaxel was purified by silica gel column chromatography with dichloromethane and methanol as the eluent (9:1). White solid, a) yield=85%, b) melting point—° C., c) HRMS (ESI): m/z [M+H]+ calcd for 1050.36338; found, 1073.3885 [Na]+. d)¹H NMR, CDCl₃, 400 MHz: δ_{H} 8.15 (s, 1H), 8.13 (s, 1H), 7.75 (s, 1H), 7.73 (s, 1H), 7.61 (t, J=7.3 Hz, 1H), 7.55-7.45 (m, 3H), 7.45-7.34 (m, 6H), 7.13 (d, J=9.0 Hz, 1H), 6.29 (s, 1H), 6.22 (t, J=8.76 Hz, 1H), 5.98 (dd, J=3.5, 9.0 Hz, 1H), 5.68 (d, J=7.04 Hz, 1H), 5.53 (d, J=3.68 Hz, 1H), 4.97 (d, J=8.24 Hz, 1H), 4.43 (dd, J=6.52 Hz, 10.8 Hz, 1H), 4.30 (d, J=8.4 Hz, 1H), 4.20 (d, J=8.3 Hz, 1H), 3.80 (d, J=7.0 Hz, 1H), 2.99-2.81 (m, 5H), 2.72 (s, 4H), 2.56-2.52 (m, 1H), 2.44 (s, 3H), 2.36-2.30 (m, 2H), 2.22 (s, 3H), 2.15-2.09 (m, 2H), 1.91 (s, 3H), 1.68 (s, 3H), 1.22 (s, 3H), 1.13 (s, 3H). ¹³C NMR, CDCl₃, 100 MHz: δ_{C} 204.16, 171.51, 170.59, 170.44, 170.10, 169.52, 168.62, 168.35, 167.77, 166.86, 157.57, 142.43, 136.72, 134.07, 133.75, 133.22, 132.19, 130.42, 129.72, 129.30, 128.83, 127.50, 127.03, 84.76, 81.35, 78.43, 76.75, 75.90, 75.25, 74.74, 72.42, 58.57, 53.00, 50.12, 49.91, 49.69, 49.48, 49.26, 49.11, 45.86, 43.49, 35.65, 34.03, 28.58, 26.86, 26.24, 25.80, 25.69, 25.15, 22.79, 22.36, 21.09, 14.88, 9.90,

3. Synthesis of BPP Peptide

The production of sample BPP (EWPRPQIPP-NH₂, SEQ ID NO:1) was carried out by solid phase peptide synthesis. Rink amide resin AM (0.222 g) was placed in a peptide synthesis tube, and 5 ml of DCM was added, followed by deprotection with piperidine, and addition of Fmoc-Pro-OH (0.135 g). After coupling with AM, the sample was deprotected with piperidine. Using the same method, this was followed by addition of Fmoc-Pro-OH (0.135 g), Fmoc-Ile-OH (0.141 g), Fmoc-Gln (Trt)-OH (0.244 g), Fmoc-Pro-OH (0.135 g), Fmoc-Arg (pdf)-OH (0.260 g), Fmoc-Pro-OH (0.135 g), Fmoc-Trp (Boc)-OH (0.211 g) and Fmoc-Glu (OtBu)-OH (0.170 g), to the peptide synthesis tube. Finally, TFA:TIS:H2O (3.8 ml:0.2 ml:0.2 ml) was added to the mixture. The peptide was cleaved from the resin to obtain a crude BPP product, and diethyl ether was added at −20° C., followed by centrifugation at 10,000 rpm for 3 min. The crude product of BPP was purified by HPLC, and analysis indicated that the polypeptide obtained was >95% pure. The molecular weight of BPP (MW: 1118.4, [M+H]+) was confirmed by ESI-MS.

4. Conjugation of 2-NHS-Succinyl-Paclitaxel to BPP Peptide

The peptide (1.2 equal) was dissolved in water and the pH of the solution was adjusted to 8.0 with sodium carbonate or triethylamine. Then, 2-NHS-succinyl-paclitaxel (1 equiv) was dissolved in acetone and added dropwise to the aqueous solution. The ratio of water and acetone was adjusted to keep the reaction mixture clear. The mixture was stirred at room temperature 25° C. for 10 min to 1 h. The reaction mixture was purified by extraction according to following procedure: After completion of the reaction, 10% acetone and ethyl acetate (EA) solution was added to the reaction mixture and the organic phase was washed 3 times with 1N HCl. NaCl was added to the mixture to improve separation of the two layers. The organic layers were combined and washed with brine (3×20 mL) and dried over Na₂SO₄. The crude product was concentrated and purified by HPLC using a Water and Methanol mixture as eluent. White solid, a) yield=88.20%, b) melting point—188-191° C., c) MS-ESI: PTX-BPP (calc—2054.25128, found—2055.0, 1027 [M]²⁺) d) ¹H NMR, (400 MHz, DMSO-d₆): δ_{H} 10.94 (t, J=14.68 Hz, 1H), 9.51 (d, J=7.76 MHz, 1H), 8.73 (s, 1H), 8.51 (s, 1H), 8.18-8.09 (m, 2H), 7.97 (d, J=7.24 Hz, 2H), 7.88 (d, J=7.16 Hz, 2H), 7.83-7.70 (m, 3H), 7.66 (t, J=7.6 Hz, 3H), 7.57-7.53 (m, 3H), 7.49-7.43 (m, 7H), 7.34 (s, 1H), 7.32 (s, 1H), 7.29 (s, 2H), 7.17-7.15 (m, 1H), 7.07-7.03 (m, 1H), 6.98 (t, J=7.52, 1H), 6.85 (s, 1H), 6.82 (s, 1H), 6.31 (s, 1H), 5.77 (t, J=7.48 Hz, 1H), 5.48 (t, J=8.84 Hz, 1H), 5.36 (dd, J=6.96, 17.64 Hz, 2H), 4.90 (d, J=9.76 Hz, 1H), 4.66 (s, 1H), 4.62 (d, J=6.24, 1H), 4.52-4.51 (m, 2H), 4.34 (t, J=6.72 Hz, 3H), 4.05-4.00 (m, 3H), 3.76 (dd, J=7.12, 14.24 Hz, 1H), 3.64-3.60 (m, 2H), 3.57-3.52 (m, 4H), 3.34 (d, J=4.76 Hz, 2H), 3.0 (d, J=4.76 Hz, 2H), 2.92-2.89 (m, 1H), 2.58 (t, J=8.28 Hz, 1H), 2.54 (s, 2H), 2.42 (t, J=7.24 Hz, 2H), 2.33 (t, J=1.8 Hz, 1H), 2.32 (s, 1H), 2.22 (s, 1H), 2.21 (s, 3H), 2.09 (s, 3H), 2.08 (s, 3H), 2.04-2.00 (m, 4H), 1.99 (s, 3H), 1.93-1.79 (m, 12H), 1.77 (s, 3H), 1.75 (s, 3H), 1.65-1.59 (m, 6H), 1.49 (s, 6H), 1.23 (s, 1H), 1.01 (s, 3H), 0.98 (s, 3H), 0.93 (d, J=7.04, 1H), 0.88 (d, J=6.44 Hz, 3H), 0.81-0.78 (m, 4H), f)¹³C NMR, (400 MHz, DMSO-d₆): δ_{c}

5. Quantitative Real-Time PCR

HEK293T, MCF-7, MDA-MB-231, and MDA-MB-468 cells were seeded in 6-well plates for 24 h, and then total RNA was isolated using RNAiso Plus Reagent (TaKaRa) according to the manufacturer's instructions. A total of 500 ng of RNA was reversely transcribed to cDNA using PrimeScript™ RT Master Mix (Perfect Real Time). Real-time PCR was performed on the Bio-Rad CFX 96 Real-time PCR system using SYBR® Premix Ex Taq™ II (Tli RNase H Plus) and specific primers. The mRNA level of each gene was normalized to β-actin with ΔΔCT method using Bio-Rad CFX Manager V1.1.308.1111 software. The oligonucleotides used in Real-time PCR were synthesized by Invitrogen, with the following sequences: ace, 5′-GCCCTGCAGGTGTCTGCAGCATGT-3′ (SEQ ID NO:2) and 5′-GGATGGCTCTCCCCGCCTTGTCTC-3′ (SEQ ID NO:3); ace2, 5′-TGGGACCACAGCGCCCGCCACTAC-3′ (SEQ ID NO:4) and 5′-TCGCCAGCCCTCCCATGCCCATAA-3′ (SEQ ID NO:5); br1, 5′-AGAGTGATCCAGGACTGCTT-3′ (SEQ ID NO:6) and 5′-GTTCAGGCAGCTGTTGACAA-3′ (SEQ ID NO:7); br2, 5′-AAGAAGTCCCGAGAGGTGTA-3′ (SEQ ID NO:8) and 5′-ACGGAGATCGAGGTTCTCAA-3′ (SEQ ID NO:9); and β-actin, 5′-AATGTCGCGGAGGACTTTGAT-3′ (SEQ ID NO:10) and 5′-AGGATGGCAAGG GACTTCCTG-3′ (SEQ ID NO:11).

6. Western Blot Analysis

Cell pellets were collected and lysed with RIPA lysis buffer containing 1×protease inhibitor cocktail and 1×phosphatase inhibitor cocktail. The protein concentration of cell samples was analyzed using the BCA method. Equal amounts of proteins from each sample were electrophoresed on 8% to 15% SDS-PAGE gels and electro transferred onto NC membranes. After incubation with appropriate primary and secondary antibodies, proteins were detected using an ECL solution and a ChemiDoc XRS+ imaging system (Bio-Rad). β-actin was used as the loading control. The intensity of the bands was analyzed using ImageJ version 1.49 software (National Institutes of Health, USA).

7. Cellular Overexpression and Knockdown of ACE

ACE siRNA (h2) (sc-270350) and Control siRNA-A (sc-37007) were purchased from Santa Cruz Biotechnology, Inc. The ACE expression plasmid was bought from Gen Script (Nanjing) Co., Ltd. Transfections were carried out using Lipofectamine 2000 (Invitrogen) according to the manufacturer's instructions.

8. Cytotoxicity Assay

MTT assays were performed to study the in vitro antitumor effects of BPP-PTX conjugate as well as free PTX and BPP in various ACE-negative and ACE-positive cell lines. Briefly, the cells (5,000 per well) were seeded in 96-well plates 24 h before treatment. The different drug formulations at concentrations of 0.01-1000 nM PTX or 0.01-1000 nM BPP-PTX and free BPP were added. The cells were cultured for another 48 h. 20 μL of 3-(4,5-dimethylthiazol-2-yl)-2,5-diphenyltetrazolium bromide (MTT) solution (5 mg/mL) was added. The cells were further cultured for 4 h, the medium was removed, and 150 μL DMSO was added to dissolve the MTT-formazan crystals. The optical density (O.D.) values for the untreated group were set as 100% viability. Independent experiments were performed in triplicate.

9. Apoptosis Assay

1×10⁵ MDA-MB-231 or 1.5×10⁵ MDA-MB-468 cells per well were cultured in 6-well plates. After attachment, cells were treated with DMSO or 10 nM PTX or BPP-PTX for 48 h and stained with Annexin V (AV) conjugated with FITC and propidium iodide (PI) using the Annexin V-FITC Apoptosis Assay Kit following the manufacturer's instructions. Stained cells were analyzed with the Cyflow Cube flow cytometer (PARTEC, Germany). Data were analyzed using FlowJo 7.6.5 software.

10. DAPI Staining

The occurrence of apoptosis was evaluated by DAPI staining. Cells (4.0×10⁵ cells/well) were seeded and incubated with 10 nM PTX or BPP-PTX for 24 h. Following treatment, the cells were washed with phosphate-buffered saline (PBS) and fixed with 4% paraformaldehyde (Sigma-Aldrich) for 15 min at room temperature 25° C., and then stained in the dark with a DAPI solution for 10 min at room temperature 25° C. Thereafter, the cells were washed with PBS and photographed under an inverted fluorescence microscope (Olympus IX71; Olympus, Tokyo, Japan).

11. Animal Study

Six-week-old female nude mice were purchased from the Laboratory Animal Services Centre, The Chinese University of Hong Kong. Mice were kept at room temperature 25° C. with an alternating 12 h light-dark cycle and were allowed access to food and water. All experimental protocols were carried out with the approval of the Committee on Use of Human and Animal Subjects in Teaching and Research of Hong Kong Baptist University and according to the Regulations of the Department of Health, Hong Kong SAR, China. MDA-MB-468 cells (5×10⁶ cells per mouse) were suspended in PBS and inoculated subcutaneously into the fourth mammary fat pad of athymic nude mice. Tumor growth was monitored every four days by measuring two perpendicular diameters with calipers. Once tumors were palpable, (˜50 mm³), mice were divided at random into four groups, each group with 8 mice, and with treatment as follows: (1) negative control group (every four days intraperitoneal injection (i.p.) PBS); (2) PTX group (every four days i.p. 2.4 μmol/kg of PTX); (3) BPP-PTX low dose group (every four days i.p. 2.4 μmol/kg of BPP-PTX); and (4) BPP-PTX high dose group (every four days i.p. 9.6 μmol/kg of BPP-PTX). The tumors were measured with calipers every day, and tumor volumes were calculated using the following formula: a²×b×0.4, where “a” is the smallest diameter and “b” is the diameter perpendicular to “a”. At the end of the experiment, the mice were sacrificed, and the tumor weights of each animal were analyzed. Blood samples (˜100 μL) were taken from the retro-orbital plexus in heparinized capillary tubes (Modulohm A/S, Herlev, Denmark). Total white blood cell counts were determined manually for each sample using a Neubauer chamber (GMBH, Wertheim, Germany).

12. Statistical Analysis

Each experiment was performed at least three times. GraphPad Prism 6.0 software was used for IC₅₀ calculations and statistical analyses, including student t-test and two-way ANOVA.

ACE is Overexpressed in TNBC Cell Lines

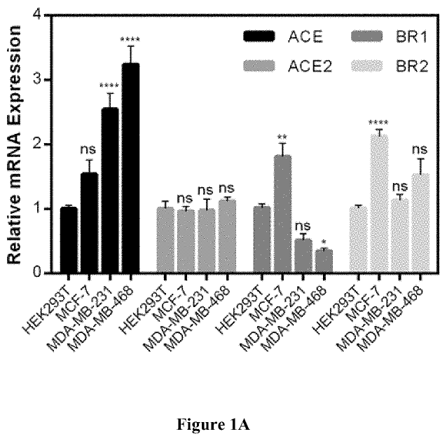

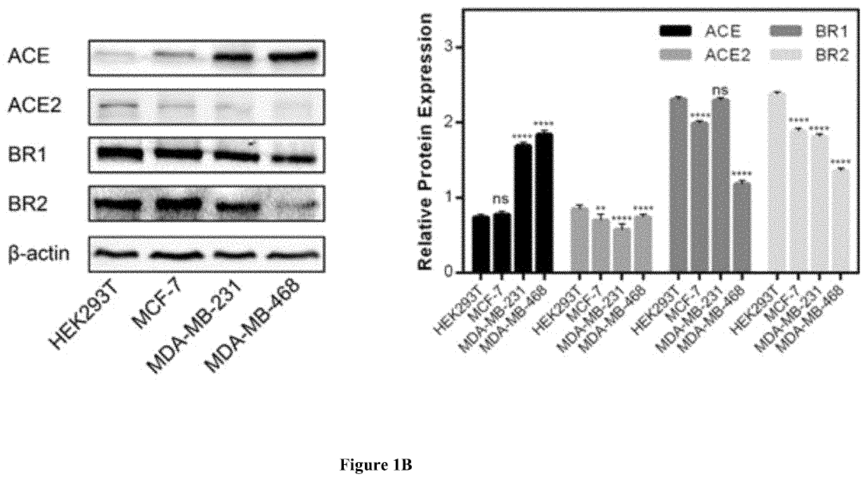

To determine whether ACE was overexpressed in TNBC cell lines or not, one human healthy kidney cell line (HEK293T) and three human breast cancer cell lines (MCF-7, MDA-MB-231, and MDA-MB-468) were used to perform the assessments. Of the breast cancer cell lines, MCF-7 is receptor-positive, while MDA-MB-231 and MDA-MB-468 are receptor-negative, and these are considered good models for TNBC. First, the mRNA expression of ACE in the aforementioned cell lines was examined. After cellular RNA was extracted and analyzed by qPCR, ACE mRNA levels were confirmed in the four cell lines. ACE mRNA levels in MDA-MB-231 and MDA-MB-468 were 2.5-fold and 3.2-fold higher than that in HEK293T (FIG. 1A). Next, the ACE protein expression profile by Western Blot was examined and it was found that ACE protein levels of MDA-MB-231 and MDA-MB-468 were also significantly higher than that of normal cell HEK-293T (FIG. 1B). These results suggested that ACE was ectopically expressed in TNBC cell lines, and ACE could be a candidate target for PDC.

Design, Synthesis, and Characterization of BPP-PTX Conjugate Targeting ACE-Positive TNBC Cell Lines

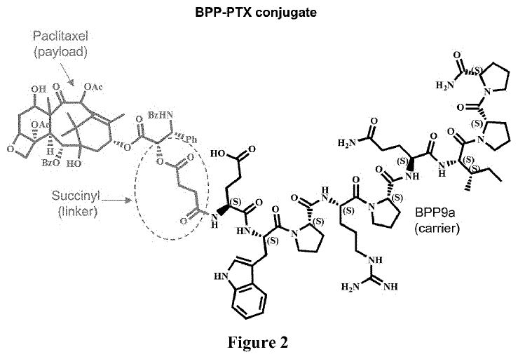

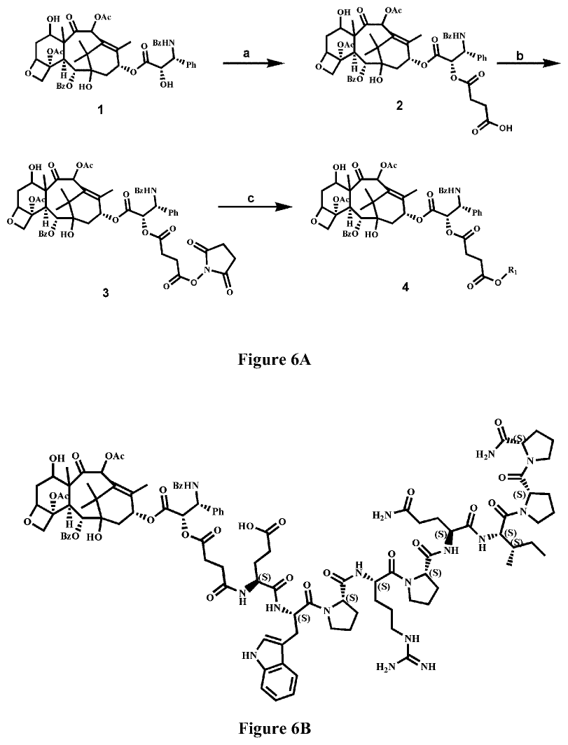

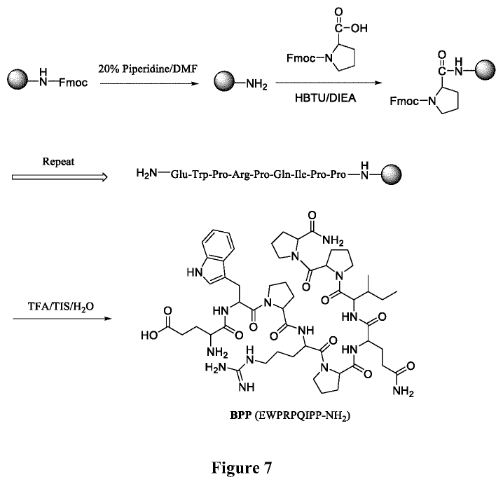

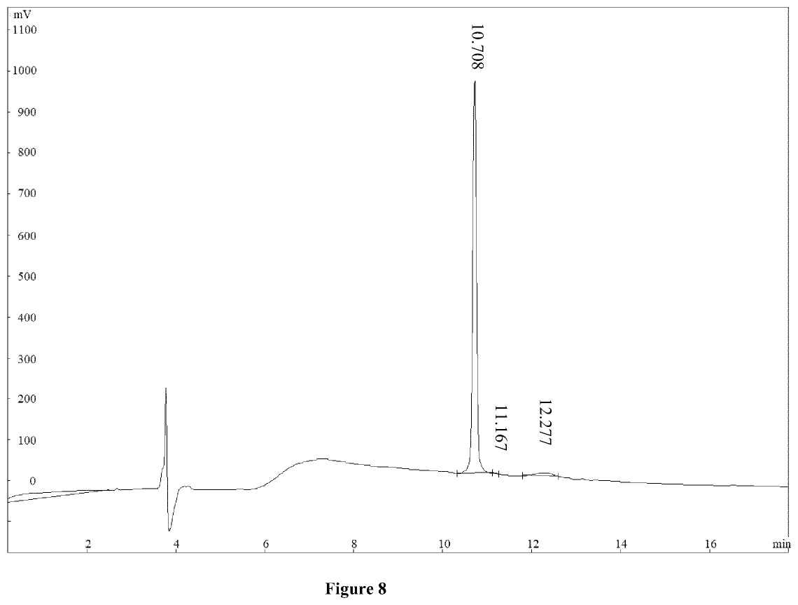

A novel PDC, BPP-PTX, in which BPP9a (EWPRPQIPP-NH₂, SEQ ID NO:1) was adopted as a carrier to target ACE, PTX was chosen as cytotoxic payload, and succinyl was used as a linker (FIG. 2). An exemplary synthetic route is presented in FIG. 6.

BPP-PTX Exhibited Potent Cytotoxicity Toward ACE-Positive TNBC Cell Lines

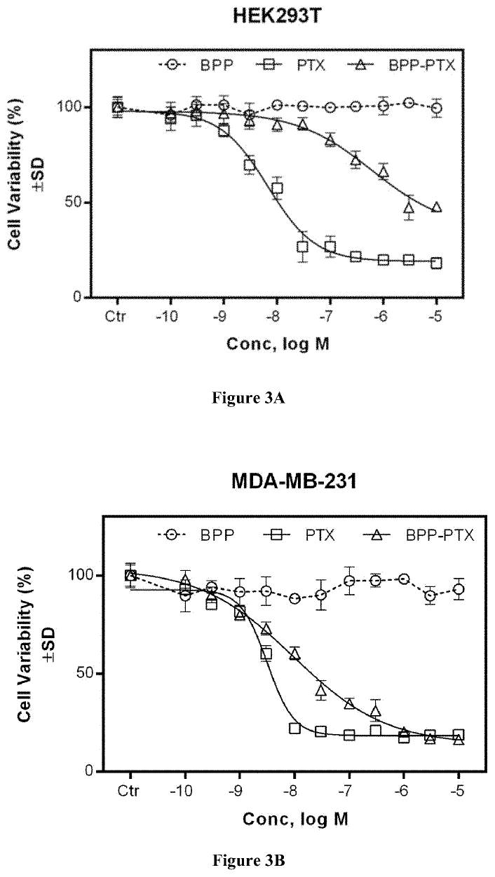

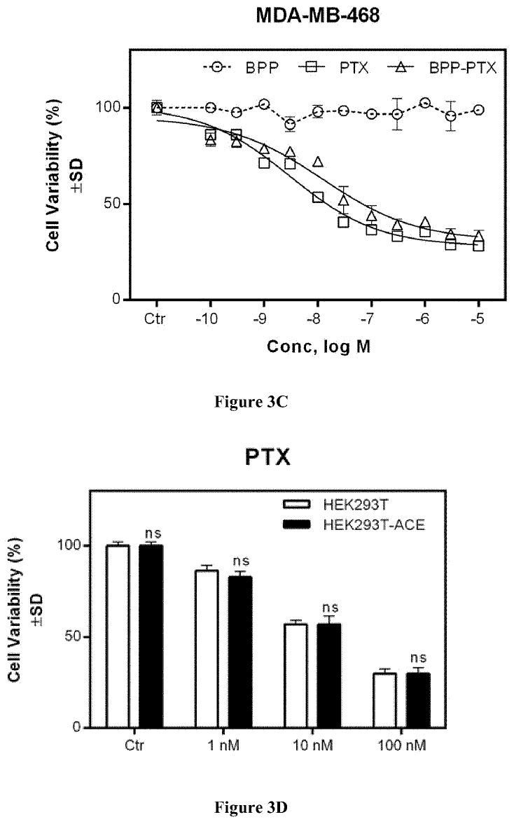

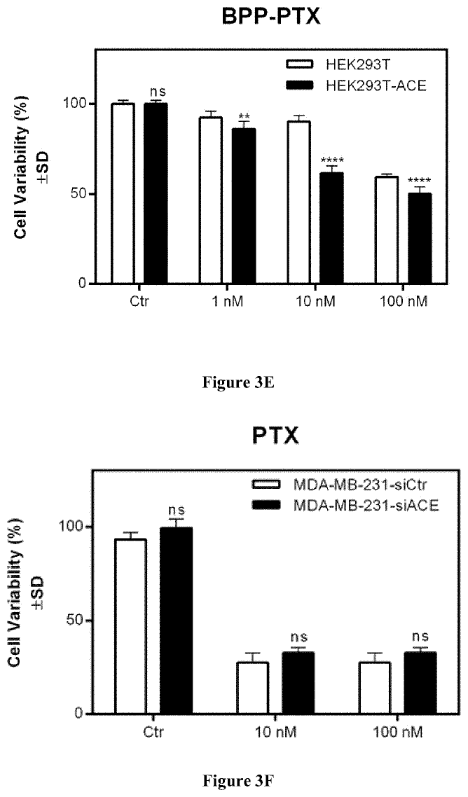

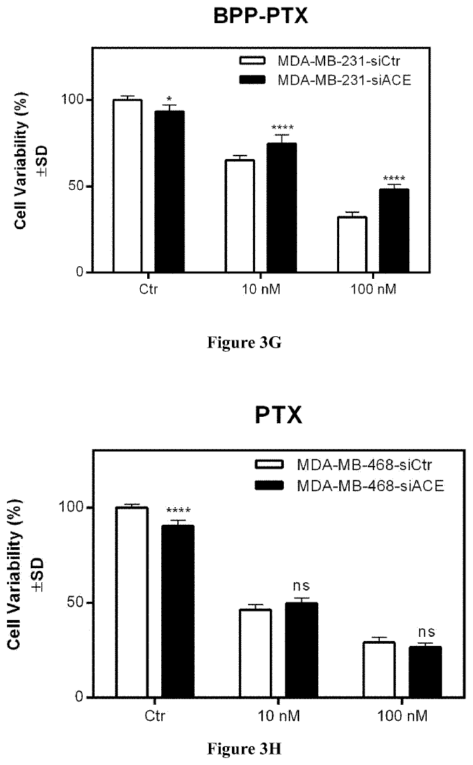

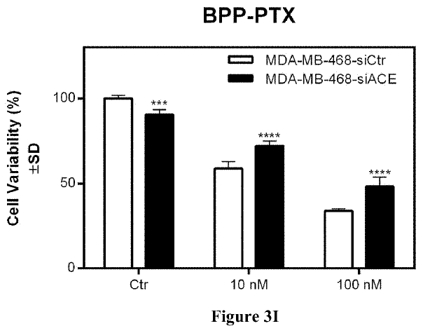

To test in vitro anti-tumor activity, the cytotoxicity of BPP-PTX was evaluated in ACE-positive TNBC cell lines (MDA-MB-231 and MDA-MB-468) and ACE-negative normal cell line (HEK293T) with MTT assay. The cytotoxicity of BPP-PTX was compared to that of the free peptide (BPP) and the free drug (PTX). The free BPP peptide exhibited no cytotoxicity in any of the three cell lines. In contrast, the IC₅₀ of BPP-PTX in ACE-negative HEK293T was 616.1 nM (95% CI, (242.7, 1564.0)), which was much higher than that of PTX in the same cell line (6.7 nM (95% CI, (5.3, 8.4))) (FIG. 3A, Table 1). Interestingly, the cytotoxicity of BPP-PTX was comparable with that of PTX in ACE-positive TNBC cell lines. In MDA-MB-231, the IC₅₀ of BPP-PTX was 9.5 nM (95% CI, (7.0, 12.8)), and the IC₅₀ of PTX was 3.1 nM (95% CI, (2.8, 3.5)). In MDA-MB-468, the IC₅₀ of BPP-PTX was 12.3 nM (95% CI, (6.8, 22.3)), and the IC₅₀ of PTX was 3.0 nM (95% CI, (2.0, 4.7)) (FIG. 3A, Table 1).

TABLE 1
Cytotoxicity of BPP, PTX, and BPP-PTX conjugates in
ACE-negative (ACE⁻) and ACE-positive (ACE⁺) cell lines.

| | IC₅₀ | | | Fold of Potency |
|---|---|---|---|---|
| | (nM)^{a} | | | Reduction |
| Cell line | BPP | PTX | BPP-PTX | (BPP-PTX:PTX) |
| HEK293T | N.D. | 6.7 (5.3, 8.4) | 616.1 (242.7, | 91.9 |
| (ACE⁻) | | | 1564.0) | |
| MDA-MB-231 | N.D. | 3.1 (2.8, 3.5) | 9.5 (7.0, 12.8) | 3.1 |
| (ACE⁺) | | | | |
| MDA-MB-468 | N.D. | 3.0 (2.0, 4.7) | 12.3 (6.8, 22.3) | 4.1 |
| (ACE⁺) | | | | |

^{a}Data were expressed as mean (95% CI).
^{b}N.D., not determined.

Next, it was determined whether the cytotoxicity of BPP-PTX was mediated by ACE or not. First, the ACE protein was artificially overexpressed in HEK293T cell lines by transfecting ACE-containing plasmid, and the empty plasmid was transfected as a control. No differences were observed in cell variability between ACE-overexpressed cells (HEK293T-ACE) or control cells (HEK293T) treated by 1-, 10-, and 100 nM PTX. However, the cell variability was significantly lower in HEK293T-ACE than in HEK293T treated with 1-, 10-, and 100 nM BPP-PTX (FIG. 3B). These results suggested that overexpression of ACE enhanced the cytotoxicity of BPP-PTX. Second, ACE was artificially knocked down in MDA-MB-231 and MDA-MB-468 cell lines by transfecting ACE siRNA (siACE), while the scrambled siRNA (siCtr) was transfected as a control. Consistently, the cell variability was of no difference in siACE or siCtr cells treated with 10-, and 100 nM PTX. However, the cell variability was significantly higher in siACE cells than in siCtr cells treated with 10- and 100 nM BPP-PTX (FIG. 3C). These results suggested that knockdown of ACE reduced the cytotoxicity of BPP-PTX. Collectively, both the overexpression and knockdown experiments supported the notion that the cytotoxicity of BPP-PTX, but not PTX, was ACE-dependent.

BPP-PTX Induces Apoptosis in ACE-Positive TNBC Cell Lines

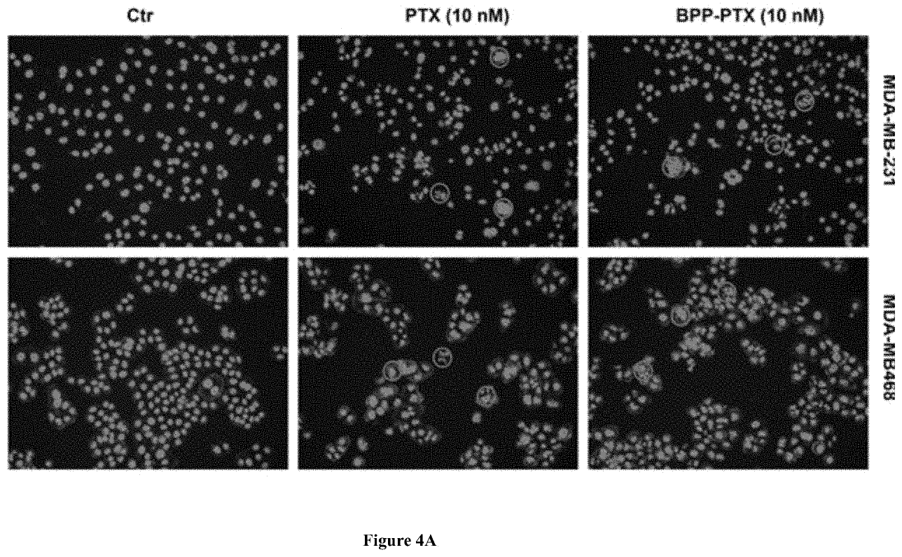

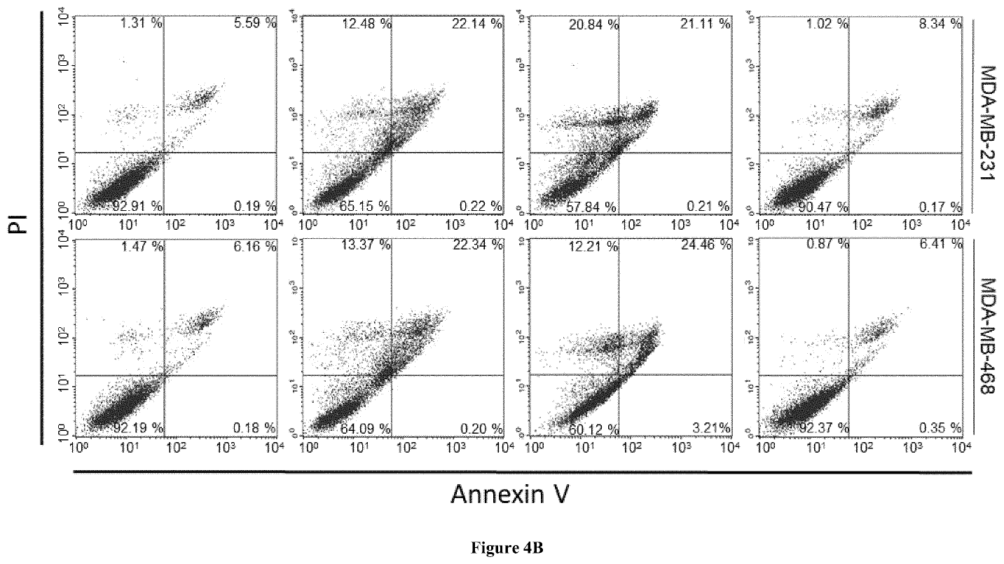

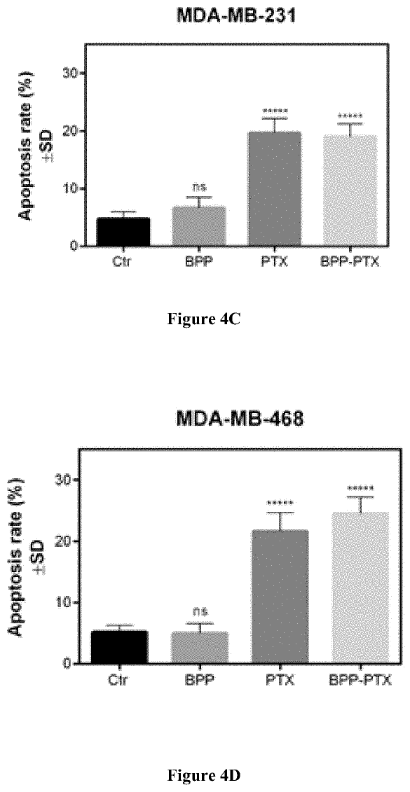

To confirm whether the cytotoxicity of BPP-PTX works through the same mechanism of PTX, morphological changes and flow cytometry analyses were performed. The nuclei of untreated control cells were large and round without condensation or fragmentation, whereas the nuclei from the 10 nM PTX treated cells were condensed and fragmented, and emitted bright fluorescence, indicative of early apoptosis (FIG. 4A). To confirm the apoptotic effects of PTX and BPP-PTX, fluorescent Annexin V-FITC/PI double staining was performed. When cells undergo apoptosis, a phosphatidylserine residue normally on the inside of the plasma membrane flips to the outside and is specifically recognized by annexin V. Counterstaining by PI allows for discrimination of apoptotic from necrotic cells. Necrotic cells were stained only with PI, whereas early apoptotic cells were stained with annexin V, and late apoptotic cells were stained with both annexin V and PI (FIG. 4B). Both PTX and BPP-PTX induced apoptosis in ACE-positive MDA-MB-468 and MDA-MB-231 cells (FIG. 4C).

BPP-PTX Suppressed Tumor Growth in TNBC Orthotopic Mouse Model with Reduced Toxicity

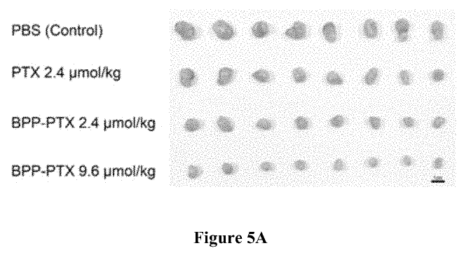

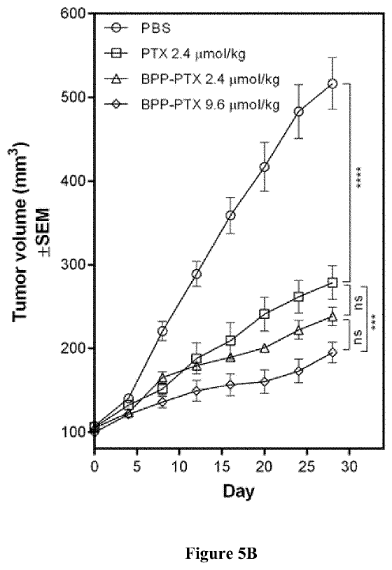

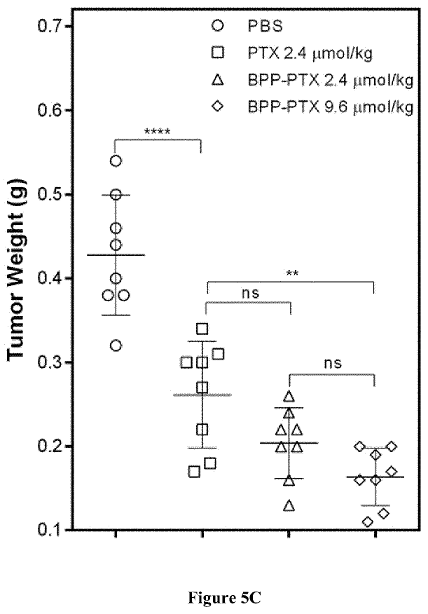

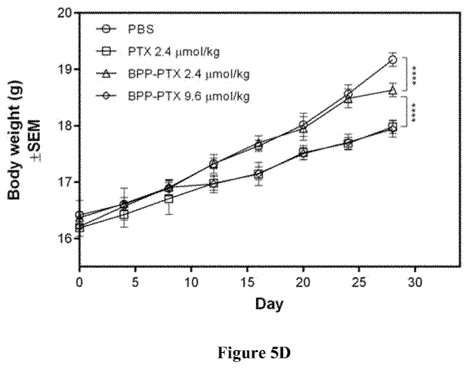

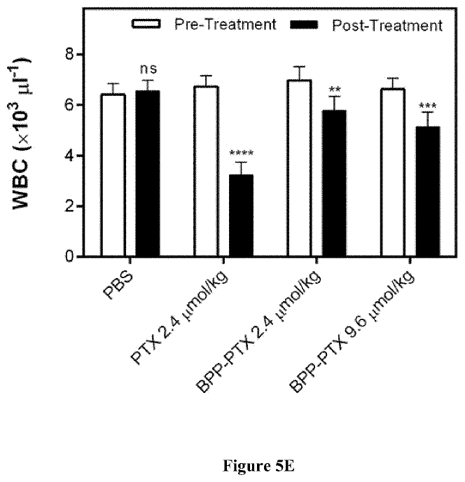

To test the in vivo efficacy and toxicity of BPP-PTX, an orthotopic mouse model was established. ACE-positive MDA-MB-468 cells were injected orthotopically in the mammary fat pads of the female athymic nude mouse. Once mice attained palpable tumors, they were randomly divided into four groups and treated for 28 days with PBS, free PTX (2.4 μmol/kg), low dose BPP-PTX (2.4 μmol/kg), and high-dose BPP-PTX (9.6 μmol/kg), every 4 days by intraperitoneal injection (i.p.). Tumor growth was monitored by measuring tumor volume every 4 days with a caliper rule. On day 4, after the first injection, the effect of the drugs was not yet detectable since average tumor volumes, as measured with the caliper, were similar in the four groups of mice. In the following days, tumor growth was significantly lower in mice treated with PTX and BPP-PTX than in control mice. On day 28, the average tumor volume of mice treated with low-dose BPP-PTX had decreased by about 15% compared to animals treated with free PTX, and by 54% compared to control animals (PBS). Meanwhile, the average tumor volume of mice treated with high-dose BPP-PTX, had decreased by about 30% compared to animals treated with free PTX, and by 62% compared to control animals (FIGS. 5A and 5B). Consistently, the average tumor weight of mice treated with low-dose BPP-PTX was 0.20 g (0.17, 0.24) lower than that of mice treated with free PTX (0.26 g (0.21, 0.31)) and control mice (0.43 g (0.37, 0.49)). The average tumor weight of mice treated with high-dose BPP-PTX was 0.16 g (0.14, 0.19), which was significantly lower than that of mice treated with free PTX and PBS (FIG. 5C). These results suggested that BPP-PTX has good tumor-suppression efficacy in vivo, even better than that of free PTX.

Body-weight-loss and white-blood-cell-reduction are well-known effects of PTX toxicity. To determine whether BPP-PTX conjugate could reduce PTX toxicity, the body weight of mice was monitored for four days during the drug treatment. Interestingly, mice treated with low-dose BPP-PTX exhibited no body weight loss from day 0 to day 24, compared with the control mice. On day 28, the body weights of mice treated with free PTX, low-dose BPP-PTX, and high-dose BPP-PTX, had decreased about 6.2%, 2.8%, and 6.4%, compared with that of control mice (FIG. 5D). The WBC count of mice treated with free PTX after the 28-day treatment had decreased about 52%, compared to their WBC counts before treatment. The WBC count of mice treated with low- and high dose BPP-PTX after the 28-day treatment had decreased about 17% and 22%, respectively. These results suggested that the BPP-PTX conjugate reduced toxicity effects of body-weight-loss and white-blood-cell-reduction, compared with that of free PTX (FIG. 5E).

PTX is considered one of the most promising cancer chemotherapy drugs and has been tested for many different human malignancies. However, clinical development has been impeded by the very high hydrophobicity of PTX and related difficulties with its formulation. Many side effects associated with treatment with PTX have also been ascribed to dilution solvents used in the formulation. Various strategies have been developed to obtain new analogs or new formulations of paclitaxel with increased cancer cell selectivity, reduced toxicity, and improved pharmacokinetic profile. Wrapping PTX with albumin protein, nanoparticle albumin bound-PTX (nab-paclitaxel) was developed and allowed to be given in water rather than in polyethoxylated castor oil by intravenous injection. Nab-paclitaxel so far is the most successful formulation of PTX. With reduced side effects caused by traditional solvents, nab-paclitaxel has been approved as a first-line therapy for metastatic TNBC. However, since albumin is not specific to tumor tissues and PTX is non-covalently bound to albumin, the toxicity caused by PTX itself is not reduced by the nab-paclitaxel formulation.

Herein, a new peptide-paclitaxel conjugate, BPP-PTX, is reported in which the drug is conjugated with the vasoactive BPP9a. BPP-PTX activity was tested in vitro in MDA-MB-231 and MDA-MB-468 cell lines, representing TNBC, and in vivo in an orthotopic mouse model of human TNBC. When conjugated with BPP peptide, paclitaxel was active against only the ACE-positive TNBC and exhibited much less activity against ACE-negative healthy cells. PTX exerts its toxicity after efficiently, but non-selectively, crossing the cell membrane, whereas BPP-PTX functioned specifically through ACE-mediated internalization in tumor cells. This difference in the internalization process may be responsible for the relatively lower in vitro efficacy of BPP-PTX, but it also confers to BPP-PTX the advantage of selectivity to tumor versus healthy cells, which cannot be appreciated in in vitro assays where only a single cell type is present.

Since BPP selectivity for cancer cells cannot be exploited in in vitro cytotoxicity tests on cancer cells alone, the cytotoxicity of BPP-PTX with the free PTX drug was examined in an in vivo animal model. Two groups of mice were injected intraperitoneally with two concentrations of BPP-PTX, a third group was injected with unconjugated PTX, and a fourth group was injected with PBS as control. Tumor growth in the four groups was subsequently different. Mice treated with PTX showed a clear reduction in average tumor growth with respect to controls, though with no tumor regression, as indicated by a positive slope of the curve obtained by plotting average tumor volume variation. Interestingly, the growth of tumors in the group of mice treated with BPP-PTX at low dose and high dose both showed a stronger tumor suppression effect with respect to the free PTX. The increase of in vivo activity of PTX induced by conjugation with BPP peptide may be related to its higher tumor enrichment. The active in vivo concentration of the free drug at the tumor site was in fact decreased by nonselective uptake by non-tumor cells. Conjugation with BPP may have mediated binding and internalization only to cells that over-express BPP membrane receptors such as ACE. Furthermore, the possible high tumor enrichment may also explain the lower toxicity associated with BPP-PTX, which would involve less PTX exposure to the normal cells.

According to the animal study, the equivalent dose of BPP-PTX in human TNBC could be estimated. The convention coefficient between mouse and human dose is 12.3 (Nair and Jacob. J Basic Clin Pharm. 2016). One can easily calculate that, the low dose of BPP-PTX for human is 0.20 μmol/kg, while the high dose of BPP-PTX for human is 0.80 μmol/kg.

Unlike PTX, BPP-PTX is very hydrophilic. This characteristic may improve its pharmacokinetic profile and enable better formulation strategy, thus further decreasing the general toxicity of chemotherapy. Compared with antibody-drug conjugates (ADCs), PDCs like BPP-PTX can be produced via chemical synthesis without fermentation, which could reduce the complexity and cost of manufacturing.

In conclusion, the present invention identified a novel surface protein, ACE, overexpressed in TNBC cell lines. Using ACE as Trojan Horse, the BPP-PTX conjugate was designed, synthesized, and evaluated. BPP-PTX exhibited potent cytotoxicity against ACE-positive TNBC cells, but weaker cytotoxicity against ACE-negative normal cells. BPP-PTX exhibited good in vivo tumor-suppression efficacy and less toxicity as characterized by body-weight-loss and WBC reduction. Conjugation with BPP improved the efficacy and safety profile of PTX. Further preclinical investigation may establish BPP-PTX as a novel targeted therapeutic for TNBC.

### INDUSTRIAL APPLICABILITY

The present disclosure relates to a new PDCs, exemplified the compound of Formula V, which were designed and synthesized by conjugating BPP9a with chemotherapeutic agent, such as a taxane, via a linker that are useful for treating cancer.

What is claimed is:

1. A peptide-drug conjugate (PDC) of Formula I:
R¹—X—R² I
or a pharmaceutically acceptable salt or zwitterion thereof, wherein
X is a linker;
R¹ is a chemotherapeutic agent;
R² is a Bradykinin-potentiating polypeptide, wherein the linker is covalently bonded to the N-terminal nitrogen of the Bradykinin-potentiating polypeptide.

2. The PDC of claim 1, wherein the chemotherapeutic agent is a taxane.

3. The PDC of claim 1, wherein the chemotherapeutic agent is paclitaxel, docetaxel, or cabazitaxel.

4. The PDC of claim 1, wherein the Bradykinin-potentiating polypeptide comprises SEQ ID NO: 1.

5. The PDC of claim 1, wherein the linker is selected from the group consisting of: *—C(═O)—**, *—(CR₂)ₙ—**, *—(CR₂)ₙCHOHCH₂—**, *—(CR₂)ₙC(═O)—**, *—C(═O)(CR₂)ₙ—**, *—C(═O)(CR₂)ₙC(═O)—**, *—C(═O)O(CR₂)ₙ—**, *—(CR₂)ₙOC(═O)—**, *—C(═O)O(CR₂)ₙOC(═O)—**, *—C(═O)N(R)(CR₂)ₙ—**, *—(CR₂)ₙ(R)NC(═O)—**, *—C(═O)(CR₂)ₙ(R)NC(═O)—**, *—C(═O)N(R)(CR₂)ₙC(═O)—**, *—C(═O)N(R)(CR₂)ₙ(R)NC(═O)—**, *—C(═O)O(CR₂)ₙ(R)NC(═O)—**, *—C(═O)N(R)(CR₂)ₙOC(═O)—**, *—(CR₂)ₙSO₂—**, *—SO₂N(R)(CR₂)ₙ—**, and *—(CR₂)ₙN(R)SO₂—**, wherein * indicates the position of a covalent bond with the chemotherapeutic agent and ** indicates the position of a covalent bond with R²; each instance of n is independently a whole number selected from 1-10; and R for each instance is independently selected from hydrogen, alkyl, cycloalkyl, and aryl; or two instances of R taken together with the carbons to which they are attached form a 3-6 membered carbocylic ring; or two instances of R taken together with the atoms to which they are attached form a 5-6 membered heterocyclic ring.

6. The PDC of claim 1, wherein the linker is *—C(═O)—**, *—C(═O)(CR₂)ₙC(═O)—**, *—C(═O)N(R)(CR₂)ₙ(R)NC(═O)—**, or *—C(═O)N(R)(CR₂)ₙC(═O)—**, wherein n is a whole number selected from 2-4.

7. The PDC of claim 1, wherein the chemotherapeutic agent has the Formula II: 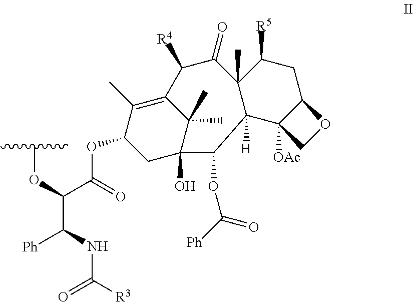
wherein R³ is phenyl, R⁴ is OAc, and R⁵ is OH; R³ is OtBu, R⁴ is OH, and R⁵ is OH; or R³ is OtBu, R⁴ is OMe, and R⁵ is OMe, wherein indicates the position of a covalent bond with X.

8. The PDC of claim 1, wherein the compound has Formula III: 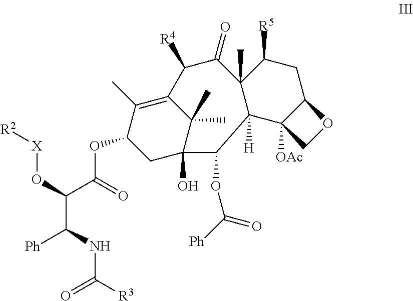
or a pharmaceutically acceptable salt or zwitterion thereof, wherein
X is a linker; and
R² is a Bradykinin-potentiating polypeptide comprising SEQ ID NO:1; and
R³ is phenyl, R⁴ is OAc, and R⁵ is OH; R³ is OtBu, R⁴ is OH, and R⁵ is OH; or R³ is OtBu, R⁴ is OMe, and R⁵ is OMe, wherein the linker is covalently bonded to the N-terminal nitrogen of the Bradykinin-potentiating polypeptide.

9. The PDC of claim 8, wherein the linker is selected from the group consisting of: *—(CH₂)ₙ—**, *—(CH₂)ₙCHOHCH₂—**, *—(CH₂)ₙC(═O)—**, *—C(═O)(CH₂)ₙ—**, *—C(═O)(CH₂)ₙC(═O)—**, *—C(═O)O(CH₂)ₙ—**, *—(CH₂)ₙOC(═O)—**, *—C(═O)O(CH₂)ₙOC(═O)—**, *—C(═O)N(H)(CH₂)ₙ—**, *—(CH₂)ₙ(H)NC(═O)—**, *—C(═O)N(H)(CH₂)ₙ(H)NC(═O)—**, *—C(═O)O(CH₂)ₙ(H)NC(═O)—**, *—C(═O)N(H)(CH₂)ₙOC(═O)—**, *—(CH₂)ₙSO₂—**, *—SO₂N(H)(CH₂)ₙ—**, and *—(CH₂)ₙN(H)SO₂—**, wherein * indicates the position of a covalent bond to a moiety of Formula IV: 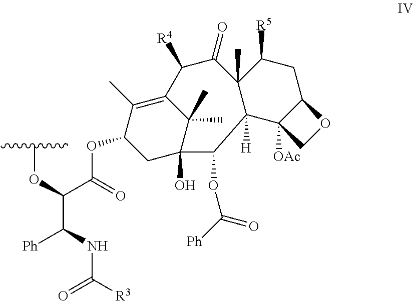
and ** indicates the position of a covalent bond with R²; and each instance of n is independently a whole number selected from 2-6.

10. The PDC of claim 9, wherein the linker is *—C(═O)(CR₂)ₙC(═O)—**, wherein n is 2-4.

11. The PDC of claim 1, wherein the compound is selected from the group consisting of: 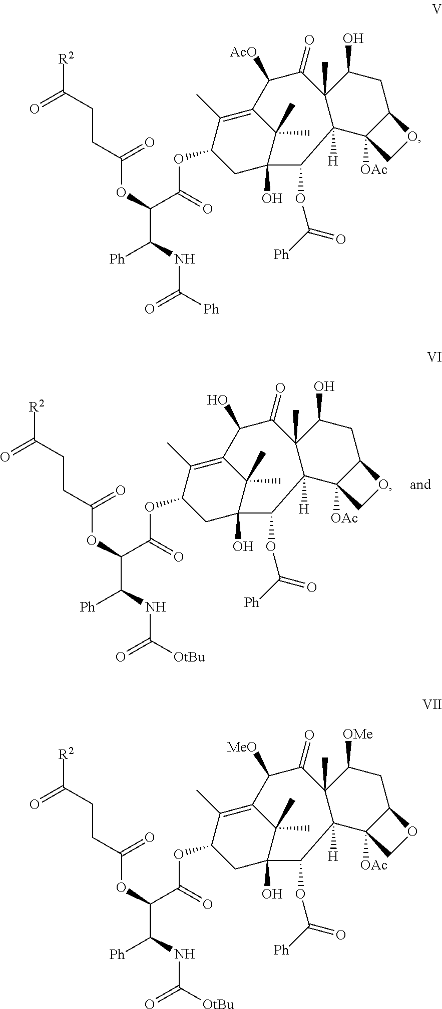
or a pharmaceutically acceptable salt or zwitterion thereof, wherein R² is SEQ ID NO:1.

12. The PDC of claim 1, wherein the compound is: 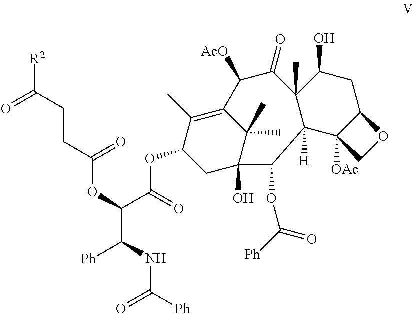
or a pharmaceutically acceptable salt or zwitterion thereof, wherein R² is SEQ ID NO:1.

13. A pharmaceutical composition comprising a PDC of claim 1 and at least one pharmaceutically acceptable excipient.

14. A method of preparing the PDC of claim 12, the method comprising: contacting a compound of Formula VIII: 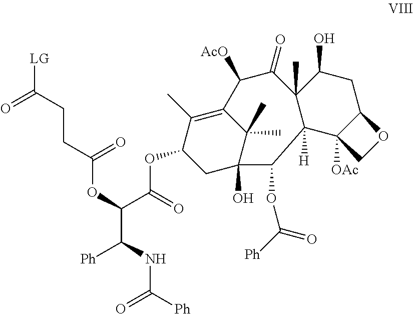
wherein LG is a leaving group; with a polypeptide comprising SEQ ID NO: 1 thereby forming the PDC of claim 12.

15. A method of treating cancer in a subject in need thereof, the method comprising: administering a therapeutically effective amount of the PDC of claim 1 to the subject.

16. The method of claim 15, wherein the cancer is selected from the group consisting of breast cancer, gastric cancer, lung cancer, head cancer, neck cancer, colon cancer, pancreatic cancer, melanoma, brain cancer, human glioblastoma, renal cancer, prostate cancer, and ovarian cancer.

17. The method of claim 15, wherein the cancer is angiotensin-converting enzyme (ACE) positive.

18. The method of claim 15, wherein the cancer is breast cancer.

19. The method of claim 15, wherein the cancer is ACE positive triple-negative breast cancer (TNBC).

20. The method of claim 15, wherein the PDC is: 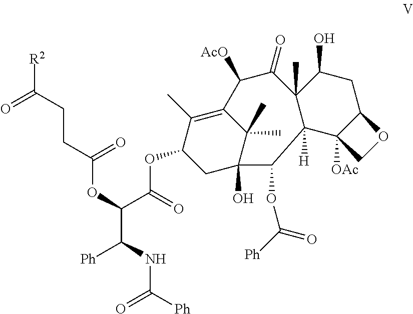
or a pharmaceutically acceptable salt or zwitterion thereof, wherein R² is SEQ ID NO:1.
